(19) United States
(12) Patent Application Publication
(10) Pub. No.: US 2011/0086374 A1
(43) Pub. Date: Apr. 14, 2011

(54) Novel Cell-Based Phosphodiesterase Assays

(76) Inventors: Jianming Lu, North Potomac, MD (US); Xiao Li, Germantown, MD (US)

(21) Appl. No.: 12/900,759

(22) Filed: Oct. 8, 2010

Related U.S. Application Data
(63) Continuation of application No. 11/709,835, filed on Feb. 23, 2007, now abandoned.
(60) Provisional application No. 60/775,786, filed on Feb. 23, 2006.

(51) Int. Cl. C12Q 1/44; C12N 9/16

(52) U.S. Cl. 435/19; 435/196

(57) ABSTRACT

The present invention relates to improved cell-based assays for the in vivo assessment of phosphodiesterase (PDE) activity using cyclic nucleotide-gated channels as cyclic nucleotide sensors, and for the assessment of the effect of PDE modulating compounds.

This application claims the benefit of Application No. 60/775,786 filed Feb. 23, 2006, the disclosure of which is hereby incorporated by reference in its entirety.

### FIELD OF THE INVENTION

The invention relates generally to cellular physiology. In particular, the invention relates to cell-based assays for measuring phosphodiesterase (PDE) activity and to screening for compounds that modulate PDE activity, such as PDE inhibitors.

### BACKGROUND OF THE INVENTION

Cyclic nucleotides are known to mediate a wide variety of cellular responses to biological stimuli. The cyclic nucleotide phosphodiesterases (PDEs) are proteins which catalyze hydrolysis of 3′,5′-cyclic nucleotides, such as cyclic adenosine monophosphate (cAMP) and cyclic guanosine monophosphate (cGMP), to their corresponding 5′-nucleotide monophosphates. These enzymes play an important role in controlling cellular concentrations of cyclic nucleotides and have a central role in a variety of intracellular signaling events, including signaling by mechanisms linked to extracellular hormones, neurotransmitters and the like.

PDEs form a superfamily of enzymes that are subdivided into 11 major families (see, for example, Beavo, Physiol. Rev. 75: 725-48, 1995; Beavo et al., Mol. Pharmacol. 46: 399-05, 1994; Soderling et al., Proc. Natl. Acad. Sci. USA 95: 8991-96, 1998; Fisher et al., Biochem. Biophys. Res. Commun. 246: 570-77, 1998; Hayashi et al., Biochem. Biophys. Res. Commun. 250: 751-56, 1998; Soderling et al., J. Biol. Chem. 273: 15553-58, 1998; Fisher et al., J. Biol. Chem. 273: 15559-64, 1998; Soderling et al., Proc. Natl. Acad. Sci. USA 96: 7071-76, 1999; and Fawcett et al., Proc. Natl. Acad. Sci. USA 97: 3702-07, 2000).

Each PDE family is distinguished functionally by unique enzymatic characteristics and pharmacological profiles. In addition, each family exhibits distinct tissue, cellular, and subcellular expression patterns (see, for example, Beavo et al., Mol. Pharmacol. 46: 399-405, 1994; Soderling et al., Proc. Natl. Acad. Sci. USA 95: 8991-96, 1998; Fisher et al., Biochem. Biophys. Res. Commun. 246: 570-77, 1998; Hayashi et al., Biochem. Biophys. Res. Commun. 250: 751-56, 1998; Soderling et al., J. Biol. Chem. 273: 15553-58, 1998; Fisher et al., J. Biol. Chem. 273: 15559-64, 1998; Soderling et al., Proc. Natl. Acad. Sci. USA 96: 7071-76, 1999; Fawcett et al., Proc. Natl. Acad. Sci. USA 97: 3702-07, 2000; Boolell et al., Int. J. Impot. Res. 8: 47-52, 1996; Ballard et al., J. Urol. 159: 2164-71, 1998; Houslay, Semin. Cell Dev. Biol. 9: 161-67, 1998; and Torphy et al., Pulm. Pharmacol. Ther. 12: 131-35, 1999). By administering a compound that selectively regulates the activity of one family or subfamily of PDE enzymes, it is possible to regulate cAMP and/or cGMP signal transduction pathways in a cell- or tissue-specific manner.

Cyclic nucleotide-gated (CNG) channels of vertebrates are cation channels controlled by the cytosolic concentration of cGMP and cAMP (for reviews, see Kaupp, 1995, Curr. Opin. Neurobiol. 5:434-442; Finn et al., 1996, Annu. Rev. Physio. 58:395-426; Zogotta and Siegelbaum, 1996, Annu. Rev. Neurosci. 19:235-263; Li et al., 1997, Q. Rev. Biophys. 30:177-193). These channels conduct cation currents, carried by mixed ions-Na⁺, K⁺ and Ca²⁺- and serve to couple both electrical excitation and Ca²⁺ signaling to changes of intracellular cyclic nucleotide concentration. In vertebrate photoreceptors and olfactory sensory receptors, CNG channels depolarize the membrane voltage and determine the activity of a number of Ca²⁺-regulated proteins involved in cell excitation and adaptation (for reviews, see Kaupp and Koch, 1992, Annu. Rev. Physiol. 54:153-175; Koch, 1995, Cell Calcium 18:314-321).

CNG channels are typically heteromultimers containing homologous α and β subunits. Some CNG channels also have a third subunit as well. For example, a third subunit has been described for the rat olfactory CNG channel (GenBank Acc. No. AF068572). Although they are members of the voltage gated channel superfamily, they are not voltage sensitive, instead responding to changes in cyclic nucleotide concentration. Modified CNG channels have been created that increase the channels' sensitivity to cAMP concentrations. See, for instance, PCT/US02/34122, PCT/US04/036,022 and Rich et al. J. 2001 J. Gen. Physiol. (118): 63-77.

G-protein-coupled receptors (GPCRs) are also of particular interest to the background of the present invention. GPCRs comprise a large super-family of integral membrane proteins characterized by having 7 hydrophobic alpha helical transmembrane (TM) domains with three intracellular and three extracellular loops (Ji, et al., J Biol Chem 273:17299-17302, 1998). In addition, all GPCRs contain N-terminal extracellular and C-terminal intracellular domains. Binding of extracellular ligand may be mediated by the transmembrane domains, the N-terminus, or extracellular loops, either alone or in combination. For example binding of biogenic amines such as epinephrine, norepinephrine, dopamine, and histamine is thought to occur primarily at the TM3 site while TM5 and TM6 provide the sites for generating an intracellular signal. Agonist binding to GPCRs results in activation of one or more intracellular heterotrimeric GTP-binding proteins (G proteins) which, in turn, transduce and amplify the signal by subsequent modulation of down-stream effector molecules (such as enzymes, ion channels and transporters). This in turn results in rapid production of second messengers (such as cAMP, cGMP, inositol phosphates, diacylglycerol, cytosolic ions).

GPCRs mediate signal transduction across a cell membrane upon the binding of a ligand to a GPCR. The intracellular portion of the GPCR interacts with a G protein to modulate signal transduction from outside to inside a cell. A GPCR is thus coupled to a G protein. There are three polypeptide subunits in a G-protein complex: an alpha subunit—which binds and hydrolyzes GTP—and a dimeric beta-gamma subunit. In the inactive state, the G protein exists as a heterotrimer of the alpha and beta-gamma subunits. When the G protein is inactive, guanosine diphosphate (GDP) is associated with the alpha subunit of the G protein. When a GPCR is bound and activated by a ligand, the GPCR binds to the G-protein heterotrimer and decreases the affinity of the G alpha subunit for GDP. In its active state, the G subunit exchanges GDP for guanine triphosphate (GTP) and active G alpha subunit disassociates from both the GPCR and the dimeric beta-gamma subunit. The disassociated, active G alpha subunit transduces signals to effectors that are “downstream” in the G-protein signaling pathway within the cell. Eventually, the G protein's endogenous GTPase activity returns active G subunit to its inactive state, in which it is associated with GDP and the dimeric beta-gamma subunit.

The transduction of the signal results in the production of second messenger molecules. Once produced, the second messengers have a wide variety of effects on cellular activities. One such activity is the activation of cyclic nucleotide-gated (CNG) channels by the cyclic nucleotides cAMP and cGMP.

Receptor function is regulated by the G protein itself (GTP-bound form is required for coupling), by phosphorylation (by O-protein-coupled receptor kinases or GRKs) and by binding to inhibitory proteins known as β-arrestins (Lefkowitz, J Biol Chem, 273:18677-18680, 1998). It has long been established that many medically significant biological processes are mediated by proteins participating in signal transduction pathways that involve G proteins and/or second messengers (Lefkowitz, Nature, 351:353-354, 1991). In fact, nearly one-third of all prescription drugs are GPCR ligands (Kallal et al., Trends Pharmacol Sci, 21:175-180, 2000).

GPCRs fall into three major classes (and multiple subclasses) based on their known (or predicted) structural and functional properties (Rana et al., Ann Rev Pharmacol Toxicol, 41:593-624, 2001; Marchese et al., Trends Pharmacol Sci, 20:370-375, 1999). Most of these receptors fall into class A, including receptors for odorants, light, and biogenic amines, for chemokines and small peptides, and for several glycopeptide/glycoprotein hormones. Class B receptors bind higher molecular weight hormones while class C includes GABA_{B} receptors, taste receptors, and Ca²⁺-sensing receptors. GPCRs are found in all tissues. However, expression of any individual receptor may be limited and tissue-specific. As such some GPCRs may be used as markers for specific tissue types.

Some evidence has suggested that at least a certain G-protein may effect signal transduction by activating some, unidentified phosphodiesterase. In particular, Ruiz-Avila et al. (Nature 1995, 376:80-85) have demonstrated that a transducin-derived peptide which mimics the effects of an activated G-protein stimulates cGMP PDE activity in bovine taste lingual-tissues. However, no direct interaction between G-protein and any particular PDE has been observed.

Given, however, the wide variety of signal transduction responses in which both G-proteins and phosphodiesterases are involved and the numerous disorders associated with these different responses, there exists a need for methods to identify specific compounds that modulate signal transduction by PDEs, G-proteins or both.

A number of PDE assays have been described. Traditionally, PDE inhibitors are screened with enzymatic assays in cell free systems. Recently, more research has been focused on cell based assays. For example, Wunder et al., (2005. Mol. Pharmacol. 68(6), 1775-1781) reported the development of a cell-based assay for a PDE9 inhibitor using a cGMP reporter cell line. In the assay, PDE9, sGC, CNGA2 and Aequorin were introduced into CHO cells. Intracellular cGMP level can be monitored via aequorin luminescence induced by Ca⁺ influx through CNG. After stimulation with submaximal concentrations of sGC activator, it was shown that PDE9 inhibitor potentiated cGMP production and caused the leftward shifts of dose response curves.

In another study, Rich et al. (J. Gen. Physiol. 118, 63-67, 2001) disclosed a cell-based assay for the in vivo assessment of local phosphodiesterase activity. The study utilized two cell lines, HEK293 and GH4C1, where wildtype and mutant CNG that has a higher affinity to cAMP were transfected into the cell. Using fluorescent calcium indicator and patch clamp to monitor CNG activity and external stimulation of forskolin or prostaglandin E1 (agonist of endogenous Gs coupled PGE receptor) to increase intracellular cAMP, the researchers were able to detect PDE inhibitor activity such as Pan-PDE inhibitor IBMX, PDE4 specific inhibitors, Ro-20-1724 and rolipram, by CNG activation.

A cell-based assay for measuring phosphodiesterase activity in which an external compound is used to stimulate intracellular cAMP production has several practical disadvantages. The optimal amount of stimulating compound for a particular PDE assay must be empirically determined. As the stimulating effect of a compound on cAMP production depends on a variety of factors, such as the growth medium, cell passage number, confluence, overall health, etc., which vary from day to day, daily determinations of the optimal amount of stimulating compound may be required to obtain reproducible results. Also, the stimulatory potency of a particular compound typically varies between suppliers and lots, and changes in potency necessitate a redetermination of the optimal amount of the compound. Furthermore, external stimulation of intracellular cAMP production may affect dose response curves of a PDE inhibitor. The complexity and need for daily recalibration of cell-based assays that use an externally provide stimulator of intracellular cAMP production make such assays undesirable for applications such a high and medium throughput screening of PDE modulating compounds.

### SUMMARY OF THE INVENTION

The present invention relates to improved cell-based assays for the in vivo assessment of phosphodiesterase (PDE) activity using intracellular cyclic nucleotide indicators that are capable of generating signals indicative of intracellular cyclic nucleotide levels, or concentrations, such as cyclic nucleotide-gated channels used with potentiometric dyes. Such indicators are useful for providing an assessment of the effect of PDE modulating compounds. The assays of the present invention provide several advantages over previously described assays, one being that the assays are carried out without the use of an externally provided stimulation of intracellular cyclic nucleotide production, such as cAMP production.

In previously described assays, an external stimulator of adenylate cyclase (AC) activity is used to increase the basal level of AC activity such that an increase in cAMP in the presence of an externally provided PDE inhibitor can be detected. Without such external stimulation, the basal level AC activity is not typically sufficient to allow detection even in the present of an externally provided PDE inhibitor. The present invention is based on the discovery that cells genetically modified to obtain a small increase in the basal level of cyclic nucleotide production, such as cAMP production, such that an increase in cyclic nucleotide in the presence of an externally provided PDE inhibitor can be detected, and such that no signal is detected in the absence of a externally provided PDE inhibitor, enable the assessment of the effect of PDE modulating compounds without the use of an externally provided stimulation of intracellular cyclic nucleotide production, particularly cAMP production.

In one aspect, for use in the present invention, cells are genetically modified to express at least one exogenously provided protein that increases the level of cyclic nucleotide production, such as cAMP production, in the absence of external stimulation of intracellular cyclic nucleotide production. Candidate modified cells may be assayed to determine the level of cyclic nucleotide production using a chosen cyclic nucleotide detection method to determine the difference in signal obtained in the presence or absence of a known PDE inhibitor. Suitable cells for use in the present invention are those which provide detectably distinct signals in the presence and absence of the known PDE inhibitor. Preferably, the cells are selected such that a detectable signal is obtained in the presence of the known PDE inhibitor, and no or little detectable signal is obtained in the absence of the PDE inhibitor.

Although the suitability of a particular modified cell will depend in part on the sensitivity and dynamic range of the chosen detection method, it can be determined routinely using a simple assay. Thus, selection of modified cells suitable for use in the present invention can be carried out routinely by screening candidate modified cells to obtain a cell possessing a level of cyclic nucleotide production, such as cAMP production, elevated appropriately to enable use with a chosen detection assay, or essentially equivalent assays. In one aspect, methods for detecting intracellular cyclic nucleotides comprise the use of intracellular cyclic nucleotide indicators that are capable of generating an optical signal indicative of a local intracellular concentration of cyclic nucleotides, particularly cAMP or cGMP, and especially cAMP. Preferably, such optical signals are based on fluorescence, chemilumenescence, bioluminescence, or the like. Exemplary intracellular cyclic nucleotide indicators include, but are not limited to, cyclic nucleotide gated (CNG) channels used in combination with one or more ion-sensitive or voltage sensitive fluorescent dyes, cyclic nucleotide-responsive genetic elements that modulate expression of a signaling molecule, e.g. luciferase, depending on cyclic nucleotide concentration, fluorescence resonance energy transfer (FRET) cyclic nucleotide indicators that generate a fluorescent signal related to cyclic nucleotide concentration, and the like. Exemplary intracellular cyclic nucleotide indicators comprising CNG channels are described more fully below and in the following references, which are incorporated by reference: U.S. Pat. Nos. 6,872,538 and 7,166,463; Rich et al, J. Gen. Physiol., 118: 63-77 (2001); Rich et al, Ann. Biomed. Eng., 30: 1088-1099 (2002); Rich et al, Methods Mol. Biol., 307: 45-61 (2005); and the like. Exemplary intracellular cyclic nucleotide indicators comprising cyclic nucleotide-responsive genetic elements, such as cAMP-responsive elements, are disclosed in Goetz et al, J. Biomol. Screen., 5: 377-384 (2000); Haizlip et al, U.S. patent publication 2003/0219825; and the like, which references are incorporated by reference. Exemplary FRET-based intracellular cyclic nucleotide indicators are disclosed in DiPilato et al, Proc. Natl. Acad. Sci., 101: 16513-16518 (2004); Nikolaev et al, J. Biol. Chem., 279: 37215-37218 (2004); Nikolaev et al, Nature Methods, 3: 23-25 (2006); and the like, which references are incorporated by reference.

In one aspect, the present invention provides methods for identifying a compound that modulates phosphodiesterase activity, comprising (a) providing a cell that expresses a cyclic nucleotide gated (CNG) channel and at least one exogenously provided protein that increases the level of cyclic nucleotide production in the absence of external stimulation of intracellular cyclic nucleotide production; (b) contacting said cell, in the absence of external stimulation of intracellular cyclic nucleotide production, with at least one compound that putatively modulates the activity of said phosphodiesterase; and (c) measuring activity of said channel, wherein changes in the activity of said channel is indicative of changes in intracellular cyclic nucleotide; thereby identifying whether said at least one putative modulatory compound modulates the activity of the PDE. A preferred cyclic nucleotide in this aspect is cAMP.

In another aspect, a method is provided for identifying a compound that modulates phosphodiesterase activity, comprising: (a) providing a cell that expresses an intracellular cyclic nucleotide indicator and at least one exogenously provided protein that increases intracellular cyclic nucleotide in the absence of external stimulation of intracellular cyclic nucleotide production to a level at or below a limit of detection of the intracellular cyclic nucleotide indicator, the intracellular cyclic nucleotide indicator being capable of generating an optical signal indicative of the level of cyclic nucleotide; (b) contacting said cell, in the absence of external stimulation of intracellular cyclic nucleotide production, with at least one compound that putatively modulates the activity of said phosphodiesterase; and (c) measuring the optical signal generated by the intracellular cyclic nucleotide indicator; thereby identifying whether said at least one putative modulatory compound modulates the activity of the phosphodiesterase. Preferably, cyclic nucleotides include cAMP or cGMP; and more preferably, the cyclic nucleotide is cAMP. In one embodiment, cells expressing an intracellular cyclic nucleotide indicator and exogenous protein are selected that express little or no optical signal in the absence of a known PDE inhibitor and a detectable signal in the presence of the known PDE inhibitor. Preferably, in such embodiments, cells are selected that produce the greatest difference in optical signal in the presence and the absence of given concentrations of such PDE inhibitor. In one aspect, a concentration of PDE inhibitor that gives rise to a detectable signal depends of the limit of detection of the intracellular cyclic nucleotide indicator employed. For example, if the limit-of-detection concentration of an indicator is close to that of the basal level of a modified host cell, then a smaller concentration of inhibitor will produce a detectable signal than otherwise would be the case. In some embodiments, the intracellular cyclic nucleotide level produced by an exogenous protein is at or near the limit of detection of an intracellular cyclic nucleotide indicator, so that in the absence of a PDE inhibitor little or not optical signal is produced, and in presence of a PDE inhibitor, intracellular cyclic nucleotide levels increase and a detectable optical signal is produced. In one embodiment, the intracellular cyclic nucleotide level produced by an exogenous protein is at the limit of detection of the selected intracellular cyclic nucleotide indicator. Preferably, the detectable optical signal is monotonically related to the intracellular concentration of cyclic nucleotide.

In preferred embodiments, the exogenously provided protein is selected from the group consisting of a G protein coupled receptor (GPCR), a G protein, and an adenylate cyclase (AC). The exogenously provided protein may be identical to an endogenous protein, and thus provide overexpression of the endogenous protein, or may be a mutant, variant, or chimeras. In some embodiments, the corresponding endogenous protein is suppressed.

In some embodiments of the invention, the cell also expresses an exogenously provided phosphodiesterase (PDE). When an exogenous PDE is expressed in the cells, it may be preferable that endogenous PDEs of the cell are suppressed.

In preferred embodiments, the cell expresses a modified cyclic nucleotide gated (CNG) channel, wherein the modification increases the sensitivity of the CNG channel to cAMP.

The cells suitable for the present invention may be derived from insect cells, amphibian cells, yeast cells, and mammalian cells. To express exogenously provided proteins, genes encoding the proteins are transfected into the cells. The transfected genes are operatively linked to promoters that are regulatable and/or heterologous. The promoters can be constitutive or inducible promoters, such as tetracycline-responsive promoters.

In some embodiments of the invention, the activity of the CNG channel is measured using an indicator selected from the group consisting of membrane potential indicators and cation-sensitive indicators. Preferably, the membrane potential indicators and cation-sensitive indicators are fluorescent dyes.

In some embodiments of the invention, control assays are carried out to compare the activation of the CNG channels in the presence of the compound that putatively modulates the PDE activity to activation of the CNG channels in the absence of the compound, wherein a difference in activation of the CNG channels indicates the compound inhibits the activity of a PDE, or to compare activation of the CNG channels in the presence of the compound to activation of the channel in the presence of a known PDE inhibitor, wherein a similar pattern of activation of the CNG channel indicates the compound inhibits the activity of a PDE.

In another aspect, the present invention provides a cell comprising a cyclic nucleotide gated (CNG) channel and at least one exogenously provided protein that increases the level of cAMP production in the absence of external stimulation of intracellular cAMP production such that activation of the CNG channel is not detected in the absence of a PDE inhibitor and wherein activation of the CNG channel is detected in the presence of a PDE inhibitor. Embodiments of the cell are as described above.

In another aspect, the present invention provides a cell produced by the steps of (a) stably transfecting host cells with an exogenous gene that encodes a protein that increases the basal level of intracellular cyclic nucleotide, where the cells express an intracellular cyclic nucleotide indicator having a limit of detection, and (b) selecting host cells that have a basal level of intracellular cyclic nucleotide level at or near the limit of detection of the intracellular cyclic nucleotide indicator. In this aspect, preferably, host cells are selected by exposing the host cells to a known concentration of a known PDE inhibitor and selecting host cells that display the greatest increase in optical signal upon such exposure. In further preference, the intracellular cyclic nucleotide indicator is a CNG channel for calcium ion and the intracellular cyclic nucleotide is cAMP.

In still another aspect, the present invention provides a kit for the identification of a modulator of a PDE that comprises a cell comprising a cyclic nucleotide gated (CNG) channel and at least one exogenously provided protein that increases the level of cAMP production without external stimulation of intracellular cAMP production such that activation of the CNG channel is not detected in the absence of a PDE inhibitor and wherein activation of the CNG channel is detected in the presence of a PDE inhibitor. Embodiments of the kits comprise a cell as described above.

### BRIEF DESCRIPTION OF THE DRAWINGS

FIG. 1 shows fluorescence intensity of ACTOne-MC1R#1 cells in the presence and absence of Ro20-1724 using a conventional potentiometric dye.

FIG. 2A is a graph showing dose-responses of PDE inhibitor Ro-20-1724 in ACTOne-MC1R#1 and ACTOne HEK293-CNG cell lines.

FIG. 2B is a graph showing dose-responses of PDE inhibitor IBMX in ACTOne-MC1R#1 and ACTOne HEK293-CNG cell lines.

FIG. 2C is a graph depicting kinetic response of ACTOne-MC1R#1 cells to Ro20-1724 using a conventional potentiometric dye.

FIG. 3A is a graph showing dose response curves of PDE inhibitor Ro20-1724 in ACTOne-TSHR#5 cell line.

FIG. 3B is a graph showing dose response curves of PDE inhibitor IBMX in ACTOne-TSHR#5 cell line.

FIG. 4 is a graph showing dose response curves of PDE inhibitors Ro20-1724 and IBMX in ACTOne-IRES-A2b#2 cell line.

FIG. 5 is a graph showing dose response curves of multiple PDE inhibitors in ACTOne-IRES-A2b#2 cell line.

FIG. 6A is a graph showing dose response curve of Ro-20-1724 in ACTOne-MC1R#1 cells using a fluorescent calcium dye reporter (BD™ PBX Calcium Assay Kit).

FIG. 6B is a graph showing dose response curve of IBMX in ACTOne-MC1R#1 cells using a fluorescent calcium dye reporter (BD™ PBX Calcium Assay Kit).

FIG. 6C is a graph depicting kinetic response of ACTOne-MC1R141 cells to Ro20-1724 using a fluorescent calcium dye reporter (BD™ PBX Calcium Assay Kit).

FIG. 7 is a graph showing the dose response curves of EHNA (A) and Bay 60-7550 (B) in PDE2A expressing ActOne-TSHR#112 cells, using a conventional potentiometric dye.

FIG. 8 is a graph showing the dose response curve of Bay 60-7550 in PDE2A and TSHR expressing ASC0200 cells, using a conventional potentiometric dye.

### DETAILED DESCRIPTION OF THE PREFERRED EMBODIMENTS

### Definitions

In the description that follows, numerous terms and phrases known to those skilled in the art are used. In the interest of clarity and consistency of interpretation, the definitions of certain terms and phrases are provided.

As used herein, cyclic nucleotide-gated (activated) ion channels include all CNG channels including those known for mediating visual and olfactory signal transductions. In native tissues, these channels are heteromultimers, with different heteromers showing distinct nucleotide sensitivity, ion conductance (selectivity), and Ca²⁺ modulation. Molecular cloning and genome sequencing efforts have revealed the presence of six genes coding for subunits of cyclic nucleotide-gated channels in human and mouse. The adopted nomenclature for these channel subunits recognizes two phylogenetically distinct subfamilies, CNGA and CNGB, defined by their sequence relationships. The CNGA subfamily comprises CNGA1 (CNG1/CNGα1/RCNC1); CNGA2 (CNG2/CNGα3/OCNC1); CNGA3 (CNG3/CNGα2/CCNC1); CNGA4 (CNG5/CNGα4/OCNC2/CNGB2). In the CNGB subfamily, the member expressed in rod photoreceptors, olfactory neurons and other tissues is designated CNGB1 (CNG4/CNGβ1/RCNC2 and a splice variant CNG4.3), whereas that found in cone photoreceptors and possibly other tissues is CNGB3 (CNG6/CNGβ2/CCNC2) (Bradley, J. et al., Nomenclature for ion channel subunits. Science. 2001 Dec. 7; 294:2095-6). It will be appreciated by those skilled in the art that channels derived from other organisms can be placed into the described subfamilies based on homology, and such channels are anticipated as applicable to the present invention.

As used herein, “voltage sensitive dyes” or “membrane potential dyes” include those dyes that enter depolarized cells, bind to intracellular proteins or membranes and exhibit enhanced fluorescence. Voltage sensitive dyes include, but are not limited to, carbocyanine, rhodamine, oxonols, and merocyanine bis-barbituric acid oxonols. Voltage sensitive and membrane potential dyes also include probes which are encoded by nucleic acid sequences that can be incorporated into a vector for expression by a host cell.

As used herein, “calcium-sensitive dyes” include those dyes which exhibit enhanced fluorescence in response to increased levels of intracellular calcium. Calcium-sensitive dyes include, but are not limited to, Fura-2, Fluo-3, Fluo-4, and Calcium Green-1. Calcium-sensitive dyes is used herein to include probes which are encoded by nucleic acid sequences that can be incorporated into a vector for expression by a host cell and include, but are not limited to, Aequorin (Euroscreen) and green fluorescent protein (GFP)-based calcium sensors such as Cameleon, for example.

As used herein, the phrase “exogenously provided,” “exogenously supplied,” or “exogenous” refers to the origin of an intracellular protein or nucleic acid. An exogenously provided nucleic acid is one that has been introduced into the cell from another source. An exogenously provided protein is one that is expressed from an exogenously provided nucleic acid. Typically, an exogenously provided nucleic acid will encode a protein that is not normally expressed within the cell, e.g., a mutant form or an analogous protein normally expressed in another species. However, an exogenously provided nucleic acid that encodes a protein identical or nearly identical town endogenous protein may be used to provide overexpression of the endogenous protein. Furthermore, characterization of a nucleic acid as exogenous does not imply any specific location of the nucleic acid after introduction into the cell. For instance, an exogenously provided nucleic acid may be expressed from a genomic or extra-genomie, chromosomal or extra-chromosomal location. Extra-genomic or extra-chromosomal locations include, but are not limited to, plasmids, viruses, and other vectors, whether they are replicative or not.

As used herein, “wildtype” proteins refer proteins having an amino acid sequence essentially identical to the protein as isolated from natural sources. As used herein, “modified,” “mutant” or “mutated” proteins refer to proteins having an altered amino acid sequence relative to the naturally occurring sequence. Alterations of the amino acid sequence may include, but are not limited to, N-terminal truncations, C-terminal truncations, amino acid residue deletions or additions, and conservative or non-conservative amino acid residue substitutions. Analogously, “modified,” “mutant” or “mutated” nucleic acids refer to nucleic acids having an altered sequence relative to the naturally occurring sequence. Alterations to nucleic acid sequences may include, but are not limited to, deletions, insertions, and substitutions.

As used herein, an “external stimulation of intracellular cAMP production” refers to the use of compound that is contacted with the cell, typically by adding to the culture medium, to increase the rate of intracelluar cAMP production. The term specifically is not meant to encompass compounds, such as PDE inhibitors, that decrease the rate of breakdown of cAMP. Examples of externally provided stimulators of intracellular cAMP production include GPCR ligands, adenylate cyclase activators, and activators of ADP-ribosylation of stimulatory G proteins.

Exemplary ligands of a GPCR include both the natural ligands and other compounds that bind to the GPCR and activate a signaling pathway that results in an increase in cAMP production.

Exemplary stimulators of ADP ribosylation include, but are not limited to, Cholera toxin (e.g., Cholergen from Vibrio cholerae, Cholera enterotoxin).

Exemplary activators of adenylate cyclase include, but are not limited to, forskolin.

As used herein, “promoter” refers to a recognition site on a DNA sequence or group of DNA sequences that provide an expression control element for a gene and to which RNA polymerase specifically binds and initiates RNA synthesis (transcription) of that gene.

As used herein, “inducible promoter” refers to a promoter where the rate of RNA polymerase binding and initiation is modulated by external stimuli. Such stimuli include light, heat, anaerobic stress, alteration in nutrient conditions, presence or absence of a metabolite, presence of a ligand, microbial attack, wounding and the like.

As used herein, “constitutive promoter” refers to a promoter where the rate of RNA polymerase binding and initiation is approximately constant and relatively independent of external stimuli.

As used herein, “inhibitors” and “antagonists” are used interchangeably throughout the application.

### PDE Assays

The present invention provides methods and cell-based assays for identifying compounds that modulate the activity of phosphodiesterases (a putative agonist or antagonist), wherein the assays comprise contacting a cell that expresses a cyclic nucleotide gated (CNG) and at least one exogenously provided protein that increases the level of cAMP production in the absence of external stimulation of intracellular cAMP with the compound, and assaying the effect of said putative agonist or antagonist compound on PDE activity. A PDE modulator compound will result, e.g., in a detectable increase or decrease in the amount of cAMP accumulation. For example, a PDE inhibitor such as Ro20-1724 or IBMX will cause an increase in cAMP levels in the cell due to inhibition of PDE and the resulting inhibition of cAMP hydrolysis. Thus, a compound can be identified as a PDE inhibitor based on its effect on an increase in intracellular cAMP. In some embodiments, it may be desirable to compare the level of intracellular cAMP in the presence of the compound to the level of intracellular cAMP in the absence of the agent. Typically, a difference in intracellular cAMP levels indicates that the agent modulates the PDE activity. Alternatively, a PDE inhibitor may also be identified based on its effect on the changes in intracellular cAMP, wherein such changes are comparable to the changes produced a known PDE inhibitor under the same assay conditions.

According to the invention, isolated primary cells or suitable cell lines are furnished with genetic material which renders them capable of expressing proteins that increase the level of cAMP production in the cells in the absence of external stimulation. For example, HEK-293 cells may be provided with a gene that encodes a GPCR such as a melanocortin 1 (MC1) receptor. The expressed or overexpressed GPCR may increase the background level of cAMP without ligand stimulation such that the increase is not sufficient to cause detectable activation of the CNG channels in the absence of a PDE inhibitor. In the present of a PDE inhibitor, levels of cAMP may be further increased, resulting in detectable activation of CNG channels. Thus, a system can be provided which allows for the measurement of the activity of a PDE inhibitor in the absence of external stimulation of cAMP production, i.e., addition of forskolin, Gs coupled receptor ligand, or other agents that act on various cellular protein components to stimulate intracellular cAMP production.

In the assays of the present invention, the exogenously provided proteins encompass any protein that is capable of increasing intracellular level of cAMP production including, among others, G protein coupled receptors (GPCR), G proteins, and adenylate cyclase (AC). The exogenously provided proteins may be mutants or variant or chimeras wherein mutation renders the proteins constitutively active to enhance cAMP production. Alternatively, the exogenously provided proteins may be expressed or overexpressed in cells wherein expression or overexpression of the exogenous proteins may also facilitate cAMP production.

### Exogenous G Protein-Coupled Receptor (GPCR)

In some embodiments, the present invention provides a host cell that contains at least a nucleic acid comprising a promoter operably linked to a polynucleotide wherein the polynucleotide comprises a sequence encoding a (GPCR) protein and a nucleic acid comprising a promoter operably linked to a polynucleotide wherein the polynucleotide comprises a sequence encoding a cyclic nucleotide-gated (CNG) channel.

The nucleic acid molecules encoding GPCRs according to the present invention may encode a full length wildtype G protein-coupled receptor or may encode a mutant GPCR. Some preferred mutants include N- and C-terminal truncations and insertion and/or deletion mutants. Other preferred mutants may have at least one conservative or non-conservative amino acid base substitution. Still other preferred mutants may have a combination of mutations, comprising at least two selected from the group consisting of N-terminal truncations, C-terminal truncations, insertions, deletions, conservative amino acid base substitutions and non-conservative amino acid base substitutions. Any GPCR may be supplied and used in the assays and methods of the invention. For instance, many GPCR sequences are publicly available, See Horn et al. In Genomics and Proteomics: Functional and Computational Aspects (Ed. S. Suhai), Kleener Academic Publishers, NV (2000), p 191-214 and Horn et al. Nucleic Acids Research (2003) 31:294-297.

### Exogenous G Protein

In some embodiments, the present invention provides a host cell that contains at least a nucleic acid comprising a promoter operably linked to a polynucleotide wherein the polynucleotide comprises a sequence encoding a G protein and a nucleic acid comprising a promoter operably linked to a polynucleotide wherein the polynucleotide comprises a sequence encoding a cyclic nucleotide-gated (CNG) channel.

The G protein may be a promiscuous G protein. The G protein may be normally expressed in the cell but may be expressed at a higher level when the cell contains the nucleic acid. Alternatively, the G protein may not be naturally expressed in the cell.

In some embodiments of the invention, the G protein-coupled receptor is substantially coupled to at least one stimulatory G protein selected from the group consisting of Gαₛ, Gα_{olf} and promiscuous G proteins. Alternatively, the G protein-coupled receptor may be substantially coupled to a hybrid G protein, such as Gα_{s/i}, for example.

It has been shown that the C-terminal 4-5 amino acids of Gα proteins encodes the domain mediating interaction with the receptor (Conklin et al. 1993. Nature 363:274-276). Chimera Gαₛ proteins in which the C-terminus of Gαᵢ proteins replaces that of a Gαₛ (Gα_{s/i}) have been shown to couple to Gαᵢ receptors, and stimulate the activity of adenylyl cyclase (Komatsuzaki et al., 1997. FEBS Letters 406:165-170).

In another preferred embodiment, at least one of the chimeric Gα, and the CNG of the current invention is stably integrated into the chromosome of the host cell. Said host cell expressing at least one of a heterologous GPCR. In yet another preferred embodiment, the chimeric Gα protein is covalently linked to the GPCR.

### Exogenous Adenylate Cyclase Protein

In some embodiments, the present invention provides a host cell that contains at least a nucleic acid comprising a promoter operably linked to a polynucleotide wherein the polynucleotide comprises a sequence encoding an adenylate cyclase (also adenylyl cyclase) protein and a nucleic acid comprising a promoter operably linked to a polynucleotide wherein the polynucleotide comprises a sequence encoding a cyclic nucleotide-gated (CNG) channel.

The exogenously provided adenylate cyclase protein is used to increase the rate of cAMP production. cAMP is produced in mammals by a family of at least nine adenylyl cyclase (AC) isozymes. The mammalian ACs differ from one another in their activation or inhibition by Ca²⁺/calmodulin, phosphorylation by protein kinases A and C, the inhibitory G protein α subunit (Gαᵢ) and the G protein β and γ subunits (Gβγ). All mammalian ACs are activated by the GTP-bound stimulatory G protein α subunit (Gαs) and all but AC9 are activated by the hypotensive drug forskolin. The known mammalian ACs consist of 12 transmembrane helices and two cytoplasmic catalytic domains (Hurley, J. H., Curr Opin Struct Biol. 1998 December; 8(6):770-7). In addition to their regulation by Gas and forskolin, mammalian adenylyl cyclases are subjected to complex regulation by other G proteins, Ca²⁺ signals, and phosphorylation.

The amount of a particular class of AC will vary between cell types. For this reason, and the above described differences in activation or inhibition, it will be appreciated that the properties of the present invention can be modified by further altering expression of at least a first adenylyl cyclase. Such alterations can include, but are not limited to, introduction of one or more heterologous ACs (both transiently expressed or integrated stably into the host genome; utilizing plasmid or viral vectors), or the up or down regulation of one or more endogenous ACs. Methods for achieving said up or down regulation are many and known to those skilled in the art. For example, cAMP production may be controlled, modulated or calibrated by the use of adenylate cyclase mutants or ACs from heterologous species, wherein said mutants or species exhibit altered levels of cAMP productions. Selection of individual mutants is within the skill of the ordinary artisan. The choice of the specific modulation is dependent upon the cell type, the G-protein linkage, the regulatory effects of various inhibitors or activators, and the means in which the present invention is to be applied.

### Exogenous CNG Channel

In the methods of the present invention, the effect of a compound on PDE activity is assessed by detecting the activity of cyclic nucleotide-gated (CNG) channels in response to changes in the intracellular cAMP. The use of CNG channels as sensors for cAMP are known in the art. (see, e.g., PCT/US02/34122, PCT/US04/036,022, U.S. Publication 2003/0157571, Rich et al, 2000, J. Gen. Physiol. 116:147-161, and Rich et al. J. 2001 J. Gen. Physiol. (118): 63-77, which are incorporated by reference herein).

The CNG channels used in the present invention may be wildtype channels, either homomeric or heteromeric, or may be or modified to make them more responsive to cAMP. In some embodiments, a modified CNG channel is used that comprises at least one mutation that makes the channel more sensitive to cAMP than a channel that does not comprise the mutation. A number of such mutations of a CNG channel α subunit that are suitable for use in the present invention are known in the art, including C460W (Gordon et al., 1997, Neuron 19:431-441), E583M (Varnum et al., Neuron 15, 619-925), and Y565A change (Li and Lester, 1998, Mol. Pharmacal. 55:873-882). In other embodiments, a modified CNG channel is used that comprises more than one mutation, such as two or three mutations, which make the channels more sensitive to cAMP than a channel that does not comprise the mutations.

Exemplary modified CNG channels for use in the present invention are described in PCT/US02/34122, PCT/US04/036,022 and Rich et al. J. 2001 J. Gen. Physiol. (118): 63-77, which are incorporated by reference herein. Cell lines stably expressing preferred modified CNG channels are commercially available from BD Biosciences (Rockville, Md.).

### Construction of Cells

The present invention further provides host cells transformed with at least one nucleic acid molecule encoding at least one exogenously provided protein. The construction of suitable cells is carried out using conventional techniques of molecular biology and nucleic acid chemistry, which are within the skill of the art and which are explained fully in the literature. See, for example, Sambrook et al., 1989, Molecular Cloning—A Laboratory Manual, Cold Spring Harbor Laboratory, Cold Spring Harbor, N.Y., which is incorporated herein by reference.

Successfully transformed cells, i.e., cells that contain an rDNA molecule of the present invention, can be identified by well known techniques including the selection for a selectable marker. For example, cells resulting from the introduction of an rDNA of the present invention can be cloned to produce single colonies. Cells from those colonies can be harvested, lysed and their DNA content examined for the presence of the rDNA using a method such as that described by Southern (J Mol Biol 98:503, 1975) or Berent et al. (Biotech 3:208, 1985) or the proteins produced from the cell assayed via an immunological method.

Preferably, the exogenous nucleic acid will be stably expressed. The creation of stable cell lines for the expression of proteins is within the capability of one ordinarily skilled in the art using standard techniques of molecular biology.

### Host Cells

Preferred cells useful for practicing the present invention include, but are not limited to, eukaryotic cells, in particular, mammalian cells. Various mammalian cell culture systems can be employed to express recombinant protein. Any cell may be used so long as the cell line is compatible with cell culture methods and compatible with the propagation of the expression vector and expression of the gene product. Preferred eukaryotic host cells include, but are not limited to, yeast, insect and mammalian cells, preferably vertebrate cells such as those from a mouse, rat, monkey or human cell line. Preferred eukaryotic host cells include Chinese hamster ovary (CHO) cells, for example those available from the ATCC as CCL61, NIH Swiss mouse embryo cells (NIH/3T3) available from the ATCC as CRL 1658, baby hamster kidney cells (BHK), mouse L cells, Jurkat cells, SF9, Xenopus oocytes, 153DG44 cells, HEK cells, PC12 cells, human T-lymphocyte cells and Cos-7 cells, ACTOne-IRES-A2b#2, ACTOne-TSHR#5, ACTone-MCIR#1 and the like eukaryotic host cells. Particularly preferred are HEK-293 cells.

Cell lines that are suitable for use in the present invention are commercially available from, for example, BD Biosciences (Rockville, Md.) or may be obtained from sources such as the ATCC (Manassas, Va.). In particular, cell lines stably expressing preferred modified CNG channels are commercially available from BD Biosciences (Rockville, Md.).

In some embodiments, the GPCR, G protein, adenylate cyclase, the CNG channel, and/or the phosphodiesterase is not normally expressed in the cell. The nucleic acids may be part of one molecule or may be parts of different molecules. The nucleic acids may be provided to the cell in any formulation known to those skilled in the art, for example, one or both of the nucleic acids may be part of a virus and/or plasmid and/or may be expressed from the genome of the cell.

### Encoding Sequences

As described above, the present invention provides recombinant DNA molecules (rDNAs) that contain a coding sequence for the aforementioned exogenously provided proteins. Gene sequences for the expression of proteins that increases the level of cAMP production are well known in the art and may be obtained from public databases such as Genbank. Preferred coding sequences are those that encode wildtype or mutant forms of one or more of GPCRs and/or G proteins, adenylate cyclase, PDE and/or CNG channels. As used herein, an rDNA molecule is a DNA molecule that has been subjected to molecular manipulation in situ. Methods for generating rDNA molecules are well known in the art, for example, see Sambrook et al., (Molecular Cloning—A Laboratory Manual, Cold Spring Harbor Laboratory Press, Cold Spring Harbor, N.Y., 1989). In the preferred rDNA molecules, a coding DNA sequence is operably linked to expression control sequences and/or vector sequences.

### Expression Constructs and Promoters

The construction of suitable expression vectors for the intracellular expression of exogenously provided genes is well known in the art. Suitable expression systems are commercially available from a large number of suppliers, such as Clontech (Moutain View, Calif.) and Invitrogen (Carlsbad, Calif.). Expression systems are available that include either constitutive, inducible, or regulatable promoters.

The choice of vector and/or expression control sequences to which one of the protein encoding sequences of the present invention is operably linked depends directly, as is well known in the art, on the functional properties desired, e.g., protein expression, and the host cell to be transformed. A vector contemplated by the present invention is at least capable of directing the replication or insertion into the host chromosome, and preferably also expression, of the structural gene included in the rDNA molecule.

Expression control elements that are used for regulating the expression of an operably linked protein encoding sequence are known in the art and include, but are not limited to, inducible promoters, constitutive promoters, and other regulatory elements. Promoters for expression in bacterial, yeast, plant or mammalian cells are known. Promoters may be added to the construct when the exogenous protein expression unit is inserted into an appropriate transformation vector, many of which are commercially available and may be obtained from suppliers such as Invitrogen (Carlsbad, Calif.), Promega (Madison, Wis.), Clontech (Moutain View, Calif.). Preferably, the inducible promoter is readily controlled, such as being responsive to tetracycline or a nutrient in the host cell's medium.

Expression vectors compatible with eukaryotic cells, preferably those compatible with mammalian cells, can be used to form rDNA molecules that contain a coding sequence. Eukaryotic cell expression vectors, including but not limited to viral vectors and plasmids, are well known in the art and are available from several commercial sources. Typically, such vectors are provided containing convenient restriction sites for insertion of the desired DNA segment. Suitable expression vectors are commercially available from a large number of suppliers, such as Clontech (Moutain View, Calif.) and Invitrogen (Carlsbad, Calif.).

Eukaryotic cell expression vectors used to construct the rDNA molecules used in the present invention may further include a selectable marker that is effective in an eukaryotic cell, preferably a drug resistance selection marker. An example of a drug resistance marker is the gene whose expression results in neomycin resistance, i.e., the neomycin phosphotransferase (neo) gene (Southern et al., J Mol Anal Genet 1:327-341, 1982). Alternatively, the selectable marker can be present on a separate plasmid, and the two vectors are introduced by co-transfection of the host cell, and selected by culturing in the appropriate drug for the selectable marker.

The nucleic acid molecule is then preferably placed in operable linkage with suitable control sequences, as described above, to form an expression unit containing the protein open reading frame. The expression unit is used to transform a suitable host and the transformed host is cultured under conditions that allow the production of the recombinant protein.

Mammalian expression vectors will typically, but not always, comprise an origin of replication, a suitable promoter and enhancer, and also any necessary ribosome binding sites, polyadenylation site, splice donor and acceptor sites, transcriptional termination sequences, and 5′ flanking nontranscribed sequences. DNA sequences derived from the SV40 splice, and polyadenylation sites may be used to provide the required nontranscribed genetic elements. Each of the foregoing steps can be done in a variety of ways. For example, the desired coding sequences may be obtained from genomic fragments and used directly in appropriate hosts. The construction of expression vectors that are operable in a variety of hosts is accomplished using appropriate replicons and control sequences, as set forth above. The control sequences, expression vectors, and transformation methods are dependent on the type of host cell used to express the gene and were discussed in detail earlier. Suitable restriction sites can, if not normally available, be added to the ends of the coding sequence so as to provide an excisable gene to insert into these vectors.

Other variants on expression vectors include fusion proteins between the gene of interest and other polypeptides. Applications include but are not limited to means of visualization (such as green fluorescent protein, GFP, and variants) or for protein purification (such as polyhistidine, or glutathione-S-transferase, GST).

Specifically contemplated are genomic DNA, cDNA, mRNA and antisense molecules, as well as nucleic acids based on alternative backbones or including alternative bases whether derived from natural sources or synthesized. Such nucleic acids, however, are defined further as being novel and unobvious over any prior art nucleic acid including that which encodes, hybridizes under appropriate stringency conditions, or is complementary to a nucleic acid encoding a protein according to the present invention.

The encoding nucleic acid molecules of the present invention may further be modified so as to contain a detectable label for diagnostic and probe purposes. A variety of such labels are known in the art and can readily be employed with the encoding molecules herein described. Suitable labels include, but are not limited to, biotin, radiolabeled nucleotides and the like. A skilled artisan can readily employ any such label to obtain labeled variants of the nucleic acid molecules of the invention.

Modifications to the primary structure of the nucleic acid molecules by deletion, addition, or alteration of the nucleotide sequence can be made without destroying the activity of the encoded proteins. Such substitutions or other alterations result in proteins having an amino acid sequence falling within the contemplated scope of the present invention.

In embodiments of the invention wherein multiple exogenously provided proteins are expressed, the encoding gene sequences may be present either on the same expression vector, or may be present on separate vectors. For example, a host cell may contain a first nucleic acid comprising a first promoter operably linked to a first polynucleotide wherein the polynucleotide comprises a sequence encoding a G protein-coupled receptor (GPCR) protein, a second nucleic acid comprising a promoter operably linked to a second polynucleotide wherein the second polynucleotide comprises a sequence encoding a cyclic nucleotide-gated (CNG) channel, and a third promoter operably linked to a third polynucleotide wherein the polynucleotide comprises a sequence encoding a phosphodiesterase (PDE) protein. These three polynucleotides may be present on the same or different expression vectors.

Any appropriate vector or expression constructs may be used to express the individual protein components in cells of the invention. For instance, such vectors may be replicable elements and replication defective elements which may insert or recombine themselves into the genome of the host cell. In some formats, promoters and/or enhancer elements may be used to control the expression levels of one or more of the proteins. In particular, the GPCR protein may be expressed from a regulated, regulatable or inducible promoter to control expression levels. Use of regulated, regulatable or inducible promoters allows for the calibration of GPCR expression levels to allow detection of putative PDE modulators or inhibitors. For instance, for certain PDE modulators or inhibitors, GPCR activity may need to be calibrated at the expression level of the protein so that adequate levels of cAMP are produced. In other instances, GPCR activity may need to be calibrated at the expression level of the protein so that cAMP levels are lowered via dampening the GPCR activity, thereby allowing detection of slight changes in cAMP concentrations.

### Transfection

Transfection of appropriate cell hosts with a suitable expression construct is accomplished by well-known methods that typically depend on the type of vector used and host system employed, With regard to transformation of vertebrate cells with DNA expression vectors, electroporation, cationic lipid or salt treatment methods are typically employed, see, for example, Graham et al. (Virol 52:456, 1973) and Wigler et al., (Proc Natl Acad Sci USA 76: 1373-1376, 1979). Transfection may also be achieved by means of retroviral infection. Similarly, a number of options are commercially available including from Invitrogen/Life Technologies (Carlsbad, Calif.), Promega (Madison, Wis.), Qiagen (Valencia, Calif.), etc.

### Screening of Stable Clones for PDE Assays

Transformed cells may be screened in standard assays to identify and select for cells that exhibit suitable balance between cAMP production or synthesis and cAMP hydrolysis. For instance, transfected or otherwise modified cells may be screened by assays to select optimum cAMP steady state levels. For example, cell colonies stably transfected with genes expressing the CNG channel, G protein, GPCR, adenylyl cyclase, and/or phosphodiesterase are screened using the following approach to identify clones for analyzing the PDE of interest without an externally provided stimulator of intracellular cAMP production. Membrane potential dye or calcium indicators can be used as detection probes as described below, and a known inhibitor of PDE of interested is used for selection. The CNG channel activity of the colonies is measured in the absence and in the presence of the known PDE inhibitor by a proper detection instrument. Colonies or cells that show different signal outputs with and without the known PDE inhibitor are desirable candidates for use in the cell-based screen for inhibitors of the PDE of interest. Typically, a change greater than about two fold in relative fluorescence or luminescence units is preferred to help achieve adequate signal to background ratio in a high-throughput screening campaign. Other controls for the selection process may include cells that do not express the PDE of interest but are identical in other aspects or cells that do not express exogenous GPCR or adenylyl cylases but are identical in other aspects. Experimental detail in colony or cell selection is described in Example 2.

### Detection

The methods of the present invention involve measuring the activity of the CNG channel, wherein changes in the activity of the CNG channel is indicative of changes in intracellular cAMP. The activity of the CNG channel can be measured using any of the methods known in the art. In preferred embodiments, activation of the CNG channel is detected by measuring either cation influx (e.g., calcium) using cation-sensitive dyes (e.g., a Ca²⁺ sensitive dye), or by measuring changes in membrane potential using voltage-sensitive dyes.

In some embodiments, cells of the present invention may be loaded with a dye that responds to the influx of Ca²⁺ with a change in one or more spectral qualities of the dye. In some embodiments, the dye binds Ca²⁺ directly resulting in an observable change in spectral quality. One example of a dye of this type is fura-2. A kit such as BD™ PBX Calcium Assay Kit (BD Biosciences, Rockville, Md.) may also be used to measure CNG channel activation. Use of calcium dyes in measuring ion channel activities is well known in the art and has been described, for example, in U.S. Pat. Nos. 5,049,673, 4,603,209, 6,162,931, 6,229,055, 5,648,270, 6,013,802, 4,795,712, PCT/US02/34122, PCT/US04/036,022, US 2003/0157571 and Rich et al. 2001 J. Gen. Physiol. (118): 63-77, which are incorporated herein by reference.

The dyes in the assay of the present invention are not limited to calcium dyes, as cAMP also induces Na⁺ and K⁺ flux in addition to Ca²⁺ changes. As a result, Na⁺ and K⁺ flux in the presence of CNG channels can be used as the indicators of intracellular cAMP accumulation. These cation-sensitive dyes are commercially available from a number of sources and their use to measure ion influx is known in the art. For example, fluorescent sodium chelators such as sodium green tetraacetate can be obtained from Molecular Probes (Eugene, Oreg.).

In other embodiments, cells may be loaded with dyes that respond to the change in membrane potential that results from the ion flux produced by the activation of the CNG channel. Dyes of this type are known to those skilled in the art (see, Zochowski, et al., 2000, Biological Bulletin 198:1-21) and are commercially available, for example, the ACTOne™ Membrane Potential Dye Kit from BD Biosciences (Rockville, Md.), the Membrane Potential Dye Kit from Molecular Devices (Sunnyvale, Calif.) and the Oxanol-Coumarin Kit from Aurora Biosciences (San Diego, Calif.). Voltage sensitive dyes that may be used in the assays and methods of the invention have been long used to address cellular membrane potentials (for review, see Zochowski et al., Biol. Bull. 198:1-21, See also U.S. Pat. Nos. 6,596,522, 6,342,379, 6,107,066, 5,661,035, 6,852,504, 6,800,765, PCT/US02/34122, PCT/US04/036,022, US 2003/0157571 and Rich et al. 2001 J. Gen. Physiol. (118): 63-77, which are incorporated herein by reference). Several classes of fluorescent dyes were developed that include carbocyanine, rhodamine, oxonols and merocyanine that can be obtained from Molecular Probes (Eugene, Oreg.). The three bis-barbituric acid oxonols, often referred to as DiBAC dyes, form a family of spectrally distinct potentiometric probes with excitation maxima at approximately 490 nm (DiBAC4(3)), 530 nm (DiSBAC2(3)) and 590 nm (DiBAC4(5)). The dyes enter depolarized cells where they bind to intracellular proteins or membranes and exhibit enhanced fluorescence and red spectral shifts (Epps et al., 1994, Chem. Phys. Lipids 69:137-150). Increased depolarization results in more influx of the anionic dye and thus an increase in fluorescence. DiBAC4(3) reportedly has the highest voltage sensitivity (Brauner et al., Biochim. Biophys. Acta. 771:208-216). Assays were developed for membrane potential assays in high throughput platforms such as FLIPR (Molecular Devices, Sunnyvale, Calif.).

As an alternative to the above-described embodiments of the cell-based assay using cation-sensitive dyes and membrane potential dyes, the present invention may also employ non-dye indicators in any of the assays described herein. For example, GFP-based indicators exist for measuring membrane potential and apoaequorin based indicators may be used to measure intracellular calcium. Aequorin is a calcium-sensitive bioluminescent protein from the jellyfish Aequorea victoria. Recombinant apoaequorin, which is luminescent in the presence of calcium but not in the absence of calcium, is most useful in determining intracellular calcium concentrations and even calcium concentrations in sub-cellular compartments. Expression vectors suitable for expressing recombinant apoaequorin and, in addition, vectors expressing apoaequorin proteins which are targeted to different sub-cellular compartments, for example the nucleus, the mitochondria or the endoplasmic reticulum are known in the art. Use of apoaequorin as a calcium indicator has been described in U.S. Pat. Nos. 5,798,441, 5,766,941, 5,744,579, 5,422,266, 5,162,227, which are incorporated herein by reference.

Other indicators are known and available for measuring other cations, such as sodium and potassium. Accordingly, the present invention may be performed using any appropriate indicator substance, including, for example, fluorescent and luminescent indicators.

Dyes of the present invention may be added exogenously to the cells either before or during the assay. Alternatively, dyes of the present invention may be expressed exogenously by the cells as probes. Said probes may be introduced into said cells for transient expression or for stable expression.

### Assay Formats and Instrumentation

The assay may be conducted by contacting a cell with a known or potential PDE modulator agent wherein the cell expresses at least one exogenously provided protein that increases cAMP production and at least one cyclic nucleotide-gated (CNG) channel including wildtype or CNGs engineered to increase the channel sensitivity to cAMP and measuring activation of the CNG channel. In some embodiments, it may be desirable to compare activation of the CNG channel in the presence of the agent to activation of the channel in the absence of the agent. Other controls configurations are known in the art. For instance, controls may include cells that do not express the GPCR of interest but are identical in other aspects. Typically, a difference in activation of the CNG channel indicates the agent modulates the activity. The CNG channel may be expressed from an exogenous nucleic acid and/or from the genome of the cell.

In some embodiments, the described invention is practiced in a multi-well plate. Standard formats include 96 well, 384 wells or 1536 wells. The disclosed invention, using intact live cells and examining cAMP levels at a single-cell level, is particularly suited for 1536 well formats. Said assays can be miniaturized to plates containing at least 1536 wells, thereby substantially reducing reagent cost, the number of cells necessary to perform the assay, and increases the throughput speed.

In some embodiments, measuring may entail determination of activation of CNG channel activity in a single cell. This may be accomplished using any means known to persons skilled in the art such as by fluorescence detection using a microscope or by flow cytometry. When a microscope is used it may be desirable to couple the microscope to a computer system. The computer system may be used to track individual cells and perform statistical analysis.

### Instruments for Fluorescence Detection

Detection of the alteration in the spectral characteristics of the dye may be performed by any means known to those skilled in the art. In preferred embodiments, the assays of the present invention are performed either on single cells using microscopic imaging to detect changes in spectral, i.e., fluorescent-properties, or are performed in a multiwell format and spectral characteristics are determined using a microplate reader.

When the assays of the invention are performed in a multiwell format, a suitable device for detecting changes in spectral qualities of the dyes used is multiwell microplate reader. Suitable devices are commercially available, for example, from Molecular Devices (FLEXstation™ microplate reader and fluid transfer system or FLIPR® system). These systems can be used with commercially available dyes such as Fluo-3, Fluo-4, and Calcium Green-1. All of these indicators excite in the visible wavelength range.

The Molecular Devices' FLIPR Fluorometric Imaging Plate Reader (Molecular Devices, Sunnyvale, Calif.) has been used in a high throughput screening assay to detect transient calcium release from intracellular with a calcium sensitive fluorescent dye in response to the activation of the Gq coupled subclass of receptors that activate the phopholipase signaling pathway

The methods of identifying an agent that modulates a PDE activity may be practiced on a single cell by determination of activation of CNG channel activity in a single cell. Methods of making such a determination are known to those skilled in the art and include by UV-based fluorescence using a microscope. When a microscope is used it may be coupled to a computer system. The computer system may be one that tracks individual cells and performs statistical analysis.

It will be apparent to those skilled in the art that it is of great utility and value that the current invention enables further reduction in the number of cells being examined, down to the single cell, and it is envisioned that screening formats with larger numbers of wells, including volumes permitting at least one cell per well, are possible. Further, the cells need not be confined to wells, rather arrays of at least one cell per feature, are envisioned. Consequently, screening formats are envisioned wherein arrays comprising hundreds, thousands, or tens of thousands of features, each feature comprising at least one cell, wherein the at least one cell expresses at least one receptor, and wherein the receptors expressed at each feature can be the same or different.

In some embodiments, the method may be configured to be conducted in a multiwell plate-96 well, 384 well etc. and measuring may be performed with a multiwell microplate reader. Examples of suitable readers include those that are fluorometric-based readers with a CCD camera and fluorometric-based scanning microplate readers.

In some embodiments, it may be desirable to attach the cells to a solid surface before, during or after performing the methods of the invention. Suitable solid surfaces include, but are not limited to, slides and multiwell plates.

### PDE Inhibitors and Inhibition

In embodiments wherein a specific type of exogenous PDE is expressed in the host cell where the PDE activity is to be measured, it is desirable to selectively suppress endogenous PDEs of other types or isozymes within cells during the assay, thereby permitting one to better characterize the PDE of interest on cAMP levels. Inhibition of PDE activity can be carried out in a number of ways, including, but is not limited to, inhibiting expression of PDE by inhibiting transcription, translation, or both, of a nucleic acid encoding PDE, or inhibiting activity of the expressed PDE protein. Inhibition of PDE isozymes may be partial or complete inhibition of PDE expression. PDE expression may be mediated by, among others, a ribozyme and/or antisense molecule that inhibits expression of a nucleic acid encoding a PDE. Inhibition of PDE activity can be effected using known PDE inhibitors specific to the isozyme, including, for example, the use of an antibody that specifically binds with PDE thereby preventing the enzyme from functioning.

An antagonist of an endogenous PDE includes molecules and compounds that prevent or inhibit the expression, activity or function of a PDE in a cell. The invention contemplates an antisense and/or antisense molecule that inhibits, decreases, and/or abolishes expression of a PDE such that the PDE is not detectable in the cell. Inhibition of endogenous PDE can be assessed using a wide variety of methods, including those disclosed herein, as well as methods well-known in the art or to be developed in the future. That is, the routineer would appreciate that inhibition of endogenous PDE expression can be readily assessed using methods that assess the level of a nucleic acid encoding endogenous PDE (e.g., mRNA) and/or the level of endogenous PDE present in a cell.

Endogenous PDE antagonist can include, but should not be construed as being limited to, a chemical compound, a protein, a peptidomemetic, an antibody, a ribozyme, and an antisense nucleic acid molecule. PDE antagonist encompasses a chemical compound that inhibits the activity of PDE. PDE antagonists are well known in the art. Additionally, PDE antagonist encompasses a chemically modified compound, and derivatives, as is well known to one of skill in the chemical arts.

Known PDE inhibitors for use in these assays herein described may be any available inhibitors in the art. For instance, PDE inhibitors include both non-specific PDE inhibitors and specific PDE inhibitors (those which inhibit a single type of phosphodiesterase with little, if any, effect on any other type of phosphodiesterase). Phosphodiesterase type V inhibitors include zaprinast, MBCQ, MY-5445, dipyridamole and sildenifil. In another embodiment, the inhibitor is a phosphodiesterase type II (PDE II) inhibitor. Suitable phosphodiesterase type II inhibitors include EHNA. In yet another embodiment, the inhibitor is a phosphodiesterase type IV (PDE4) inhibitor, Suitable phosphodiesterase type IV inhibitors include ariflo (SB207499), RP73401, CDP840, rolipram and LAS31025. In yet another embodiment, the inhibitor is a nonspecific phosphodiesterase (nonspecific PDE) inhibitor. Suitable nonspecific phosphodiesterase inhibitors include IBMX, theophylline, aminophylline, pentoxifylline, papaverine and caffeine.

Exemplary PDE antagonists include, but are not limited to, theophylline (e.g., 3,7-dihydro-1,3-dimethyl-1H-purine-2,6-dione; 2,6-dihydroxy-1,3-dimethylpurine; 1,3-dimethylxanthine); caffeine (e.g., 1,3,7-trimethylxanthine); quercetin dihydrate (e.g., 2-(3,4-dihydroxyphenyl)-3,5,7-trihydroxy-4H-1-benzopyran4-one dihydrate; 3,3′,4′,5,7-pentahydroxyflavone dihydrate); rolipram (e.g., 4-[3-(cyclopentyloxy)-4-methoxyphenyl]-2-pyrrolidinone); 4-(3-butoxy-4-methoxybenzyl)imidazolidin-2-one; propentofylline (e.g., 3,7-dihydro-3-methyl-1-(5-oxohexyl)-7-propyl-1H-purine-2,6-dione; 3-methyl-1-(5-oxohexyl)-7-propylxanthine); 3-Isobutyl-1-methylxanthine (e.g., 3,7-dihydro-1-methyl-3-(2-methylpropyl)-1H-purine-2,6-dione; IBMX; 3-isobutyl-1-methyl-2,6(1H,3H)-purinedione; 1-methyl-3-isobutylxanthine); 8-Methoxymethyl-3-isobutyl-1-methylxanthine (e.g., 8-methoxymethyl-IBMX); enoximone (e.g., 1,3-dihydro-4-methyl-5-[4-methylthiobenzoyl]-2H-imidazol-2-one); papaverine hydrochloride (e.g., 6,7-Dimethoxy-1-veratrylisoquinol-ine hydrochloride).

Other exemplary PDE inhibitors include, but are not limited to: calmidazolium chloride (e.g. 1-[bis(4-chlorophenyl)methyl]-3-[2,4-dichloro-b-(2,4-dichlorobenzyloxy)phenethyl]imidazolium chloride; 1-[bis(4-chlorophenyl)methyl]-3-[2-(2,4-dichlorophenyl)-2-(2,4-dichlorobenzyloxy)ethyl]-1H-imidazolium chloride); SKF 94836 (e.g., N-cyano-M-methyl-N″-[4-(1,4,5,6-tetrahydro-4-methyl-6-oxo-3-pyridazinyl)-phenyl]guanidine; Siguazodan); neuropeptide Y fragment 22-36 (e.g., Ser-Ala-Leu-Arg-His-Tyr-Ile-Asn-Leu-Ile-Thr-Arg-Gln-Arg-Tyr; aminophylline hydrate (e.g., 3,7-Dihydro-1,3-dimethyl-1H-purine-2,6-dione-, compound with 1,2-ethanediamine (2:1) (Theophylline)₂; ethylenediamine; theophylline hemethylenediamine complex); butein (e.g., 1-(2,4-dihydroxyphenyl)-3-(3,4-dihydroxyphenyl)-2-propen-1-one; 2′,3,4,4′-tetrahydroxychalcone); papaverine hydrochloride (e.g., 6,7-dimethoxy-1-veratrylisoquinoline hydrochloride); etazolate hydrochloride (e.g., 1-ethyl-4-[(1-methyl ethylidene)hydrazino]1H-pyrazolo[3,4-b]pyridine-5-carboxylic acid ethyl ester hydrochloride); trifluoperazine dihydrochloride (e.g., 10-[3-(4-methyl-1-piperazinyl)prop-yl]-2-trifluoromethyl-phenothiazine dihydrochloride; trifluoroperazine dihydrochloride); and milrinone (e.g., 1,6-Dihydro-2-methyl-6-oxo-(3,4′-bipyridine)-5-carbonitrile).

Particularly preferred are selective inhibitors specific for PDE4. Many known selective PDE4 inhibitors fall into one of six chemical structural classes, rolipram-like, xanthines, nitraquazones, benzofurans, naphthalenes and quinolines. Examples of rolipram-like analogs include imidazolidinones and pyrrolizidinone mimetics of rolipram and Ro 20-1724, as well as benzamide derivatives of rolipram such as RP 73401 (Rhone-Poulenc Rorer). Xanthine analogs include Denbufylline (SmithKline Beecham) and Arofylline (Almirall); Nitraquazone analogs include CP-77,059 (Pfizer) and a series of pyrid[2,3d]pyridazin-5-ones (Syntex); Benzofuran analogs include EP-685479 (Bayer); Napthalene analogs include T-440 (Tanabe Seiyaku); and Quinoline analogs include SDZ-ISQ-844 (Novartis).

PDE antagonist encompasses an antibody that specifically binds with a PDE isomer thereby blocking the interaction between the PDE isomer and its ligands. Antibodies to a PDE isomer can be produced using standard methods disclosed herein or well known to those of ordinary skill in the art (Harlow et al., 1988, Antibodies: A Laboratory Manual, Cold Spring Harbor, N.Y.). Thus, the present invention is not limited in any way to any particular antibody; instead, the invention includes any antibody that specifically binds with the PDE isomer either known in the art and/or identified in the future.

An antibody can be administered as a protein, a nucleic acid construct encoding a protein, or both. Numerous vectors and other compositions and methods are well known for administering a protein or a nucleic acid construct encoding a protein to cells or tissues. Therefore, the invention includes a method of administering an antibody or nucleic acid encoding an antibody (e.g., synthetic antibody) that is specific for a PDE isomer. (Sambrook et al., 1989, Molecular Cloning: A Laboratory Manual, Cold Spring Harbor Laboratory, New York; Ausubel et al., 1997, Current Protocols in Molecular Biology, John Wiley & Sons, New York).

The invention encompasses administering an antibody that specifically binds with a PDE isomer of interest, or a nucleic acid encoding the antibody. Such antibodies, frequently referred to as “intrabodies”, are well known in the art and are described in, for example, Marasco et al. (U.S. Pat. No. 6,004,490) and Beerli et al. (1996, Breast Cancer Research and Treatment 38:11-17). Thus, the invention encompasses methods comprising blocking the binding of PDE ligands to PDE or inhibiting expression of PDE on a cell.

The present invention is not limited to chemical compounds and antibodies against PDE. One of skill in the art would appreciate that inhibiting the expression of a polypeptide is like wise an effective method of inhibiting the activity and function of the polypeptide. Thus, a method is provided for the inhibition of a PDE isomer by inhibiting the expression of a nucleic acid encoding a PDE isomer. Methods to inhibit the expression of a gene are well known to those of ordinary skill in the art, and include the use of ribozymes or antisense oligonucleotide.

Antisense oligonucleotides are DNA or RNA molecules that are complementary to some portion of an mRNA molecule. When present in a cell, antisense oligonucleotides hybridize to an existing mRNA molecule and inhibit translation into a gene product. Inhibiting the expression of a gene using an antisense oligonucleotide is well known in the art (Marcus-Sekura, 1988, Anal. Biochem. 172:289), as are methods of expressing an antisense oligonucleotide in a cell (Inoue, U.S. Pat. No. 5,190,931).

Contemplated in the present invention are antisense oligonucleotides that are synthesized and provided to the cell by way of methods well known to those of ordinary skill in the art. As an example, an antisense oligonucleotide can be synthesized to be between about 10 and about 100, more preferably between about 15 and about 50 nucleotides long. The synthesis of nucleic acid molecules is well known in the art, as is the synthesis of modified antisense oligonucleotides to improve biological activity in comparison to unmodified antisense oligonucleotides (Tullis, 1991, U.S. Pat. No. 5,023,243).

Similarly, the expression of a gene may be inhibited by the hybridization of an antisense molecule to a promoter or other regulatory element of a gene, thereby affecting the transcription of the gene. Methods for the identification of a promoter or other regulatory element that interacts with a gene of interest are well known in the art, and include such methods as the yeast one hybrid system (Bartel and Fields, eds., In: The Yeast Two Hybrid System, Oxford University Press, Cary, N.C.).

Alternatively, reduction or inhibition of a gene expressing PDE can be accomplished through the use of a RNA interference (RNAi). As is well known to those skilled in the art, this is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. See, for example, U.S. Pat. No. 6,506,559; Fire et al., Nature (1998) 391(19):306-311; Timmons et al., Nature (1998) 395:854; Montgomery et al., TIG (1998) 14(7):255-258; David R. Engelke, Ed., RNA Interference (RNAi) Nuts & Bolts of RNAi Technology, DNA Press (2003); and Gregory J. Hannon, Ed., RNAi A Guide to Gene Silencing, Cold Spring Harbor Laboratory Press (2003). Therefore, the present invention also includes methods of silencing the gene encoding PDE by using RNAi technology.

Alternatively, reduction or inhibition of a gene expressing PDE can be accomplished through the use of a ribozyme. Using ribozymes for inhibiting gene expression is well known to those of skill in the art (see, e.g., Cech et al., 1992, J. Biol. Chem. 267:17479; Hampel et al., 1989, Biochemistry 28: 4929; Altman et al., U.S. Pat. No. 5,168,053).

Antagonists of PDE gene expression can be administered singly or in any combination thereof. Further, PDE antagonists can be administered singly or in any combination thereof in a temporal sense, in that they may be administered simultaneously, before, and/or after each other.

### Kits

The present invention further provides kits adapted to perform the methods of the invention. Such kits will typically include one or more cells of the invention in a suitable container. Kits may optionally comprise one or more reagents such as buffers and/or salts and/or dyes. When dyes are included, they will typically be voltage sensitive dyes and/or Ca²⁺ sensitive dyes.

Agents that Modulate PDE Activity

Potential agents can be screened to determine if application of the agent modulates a PDE-mediated activity. This may be useful, for example, in determining whether a particular drug is effective in treating a particular patient with a disease characterized by an aberrant PDE-mediated activity. In the case where the activity is affected by the potential agent such that the activity returns to normal or is altered to be more like normal, the agent may be indicated in the treatment of the disease. Similarly, an agent that induces an activity that is similar to that expressed in a disease state may be contraindicated.

According to the present invention, a PDE with a known inhibitor may be used as the basis of an assay to evaluate the effects of a candidate drug or agent on a cell, for example on a diseased cell. A candidate drug or agent can be screened for the ability to modulate an activity mediated by the PDE, for example Ca²⁺ influx.

Agents that are assayed in the above methods can be randomly selected or rationally selected or designed. As used herein, an agent is said to be randomly selected when the agent is chosen randomly without considering the specific sequences involved in the association of the a protein of the invention alone or with its associated substrates, binding partners, etc. An example of randomly selected agents is the use a chemical library or a peptide combinatorial library, or a growth broth of an organism.

As used herein, an agent is said to be rationally selected or designed when the agent is chosen on a nonrandom basis which takes into account the sequence of the target site and/or its conformation in connection with the agent's action. Agents can be rationally selected or rationally designed by utilizing the peptide sequences that make up these sites. For example, a rationally selected peptide agent can be a peptide whose amino acid sequence is identical to or a derivative of any functional consensus site.

The agents of the present invention can be, as examples, peptides, small molecules, vitamin derivatives, as well as carbohydrates, lipids, oligonucleotides and covalent and non-covalent combinations thereof. Dominant negative proteins, DNA encoding these proteins, antibodies to these proteins, peptide fragments of these proteins or mimics of these proteins may be introduced into cells to affect function. “Mimic” as used herein refers to the modification of a region or several regions of a peptide molecule to provide a structure chemically different from the parent peptide but topographically and functionally similar to the parent peptide (see Grant, (1995) in Molecular Biology and Biotechnology Meyers (editor) VCH Publishers). A skilled artisan can readily recognize that there is no limit as to the structural nature of the agents of the present invention.

Uses for Agents that Modulate PDE Activity

Agents that modulate one or more PDE activities, such as agonists or antagonists of a PDE may be used to modulate processes associated with PDE function and activity. In some embodiments, agents that modulate a PDE-mediated activity-increase, decrease, or change the kinetic characteristics of the activity may be used to modulate biological and pathologic processes associated with one or more PDE activity.

As used herein, a subject can be any vertebrate, preferably a mammal, so long as the vertebrate or mammal is in need of modulation of a pathological or biological process mediated by a PDE protein of the invention. The term “mammal” is defined as an individual belonging to the class Mammalia. The invention is particularly useful in the treatment of human subjects.

Pathological processes refer to a category of biological processes that produce a deleterious effect. For example, a particular PDE-mediated activity or level of activity may be associated with a disease or other pathological condition. As used herein, an agent is said to modulate a pathological process when the agent reduces the degree or severity of the process.

The agents of the present invention can be provided alone, or in combination with other agents that modulate a particular pathological process. For example, an agent of the present invention can be administered in combination with other known drugs. As used herein, two agents are said to be administered in combination when the two agents are administered simultaneously or are administered independently in a fashion such that the agents will act at the same time.

The agents of the present invention can be administered via parenteral, subcutaneous, intravenous, intramuscular, intraperitoneal, transdermal, or buccal routes. Alternatively, or concurrently, administration may be by the oral route. The dosage administered will be dependent upon the age, health, and weight of the recipient, kind of concurrent treatment, if any, frequency of treatment, and the nature of the effect desired.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### EXAMPLES

### Example 1

### Establishment of Stable Transfection Colonies for PDE Assay

Various mammalian cell culture systems can be employed to express recombinant protein. Examples of mammalian expression systems include the COS-7 lines of monkey kidney fibroblasts, described by Gluzman (Cell 23:175, 1981), and other cell lines capable of expressing a compatible vector, for example, the C127, 3T3, CHO, HeLa and BHK cell lines.

In this example, cell lines that stably express recombinant proteins were generated. Following the protocol recommended by InVitrogen Corporation (Carlsbad, Calif.), BD ACTOne™ HEK293-CNG cells (HEK293H cell stably expresses a mutant CNG channel, Cat # 341467, BD Biosciences, Rockville, Md.) were first split into 6-well plates with 70-80% confluence. For each well, the cells were transfected with 2 μg pCMV-A2b-IRES-PURO (plasmid for overexpressing human adenosine A2b receptor using CMV promoter) and 6.25 μl Lipofectamine 2000 (InVitrogen Corporation). About 18-22 hrs later, the transfected cells were split. Cells in each well were placed into two 10 cm dishes and diluted properly. The cells were selected in the medium of DMEM 10% FBS supplemented with 250 μg/ml G418 and 2 μg/ml Puromycin. The medium may be changed if there are too many dead cells. After about 2-3 weeks, the colonies could be observed. Single colonies were selected and transferred into 24 well-plates containing 1 ml of DMEM-10% FBS supplemented with 250 μg/ml G418 and 1 μg/ml Puromycin.

To generate cell lines that stably express human melanocortin 1 receptor (MC1R), and human thyroid stimulating hormone receptor (TSHR) by viral infection, Phoenix-ampho cells were plated at about 2/3 confluence 18-24 hours prior to transfection, in 10 cm dishes (approximately 5 million cells per dish) in 15 ml of DMEM with 10% PBS.

On the day of transfection (day 1), 20 μg of DNA (pBabe-MC1R and pBabe-TSHR, viral vectors for overexpressing MC1R and TSHR using a retro-viral promoter) in 1.5 ml of Opti-MEM (Invitrogen) was diluted. Also diluted in 1.5 ml of Opti-MEM was 40 μl of Lipofectamine 2000 (Invitrogen). The diluted Lipofectamine 2000 was kept at room temperature for 5 min and then combined with the diluted DNA within 30 min. Longer incubation decreases activity. The mixture of DNA and Lipofectamine 2000 was incubated at room temperature for 20 min to allow DNA-Lipofectamine complexes to form. The DNA-Lipofectamine 2000 complexes were added directly to Phoenix-Ampho Cells in the dishes which were gently rocked back and forth to facilitate mixing. The cells were then incubated in a 37° C. CO₂ incubator overnight and the media in the cell plates were replaced by 3 ml fresh DMEM, 10% FBS 24 hours post transfection (day 2). In the meantime, ACTOne HEK293-CNG cells were divided at a concentration of 1×10⁶ cells per 10 cm dish in 10 mls of DMEM with 10% FBS for infection.

In the morning of Day 3, the media of ACTOne HEK293-CNG cells were replaced with 3 ml of fresh warmed media. 3 nits of supernatants from transfected Phoenix cells were transferred into a 15 ml conical tube. 3 mls of fresh media were added to the transfected Phoenix cells and the cells were placed in a 37° C. CO₂ incubator. The supernatants were filtered with 0.45 μM filters and added to the dishes containing ACTOne HEK293-CNG cells for infection. The dishes were placed in a 37° C. CO₂ incubator. In the evening, the steps of infection were repeated except that no more fresh media were added to transfected Phoenix cells.

In the morning of day 4, the media of infected cells were replaced with fresh DMEM, 10% FBS, 1×Pen/Strep. In the evening, the cells were selected with DMEM −10% FBS supplemented with 250 μg/ml G418 and 2 μg/ml Puromycin. Continued with selection by changing media every 3-4 days, colonies were observed 2-3 weeks after selection, Single colonies were selected and transferred into 24 well-plates containing 1 ml of DMEM-10% FBS supplemented with 250 μg/ml G418 and 1 μg/ml Puromycin.

### Example 2

### Identification of Stable Cell Lines for PDE Assay

Transfection colonies obtained by approaches described in Example 1 can be screened by the following method to identify clones that are suitable for PDE assays. Stable clones with HEK293 background can be selected for PDE IV assays without introducing exogenous PDE IV gene, since PDE IV is the most abundant PDE isozyme in HEK293.

When cell density of colonies in 24-well plates (described in Example 1) reaches 60-80% confluence, remove culture medium and replace it with 1 ml of Dulbecco's Phosphate Buffers Saline without calcium and magnesium (DPBS). Remove DPBS and add 75 μl of 1× trypsin-EDTA to each well. Rock the plate to make sure the cells are equally covered with the solution. Incubate at room temperature for 5 min. Add 180 μl of growth medium (DMEM with 10% FBS, 250 μg/ml G418 and 1 μg/ml Puromycin) into each well. Suspend the cells well and plate 20 μl/well of cell suspension into a poly-D-lysine-coated 384-well assay. Plate multiple wells for each clone.

After overnight incubation, cell plates are removed from the incubator. 20 μl 1× ACTOne Membrane Potential Dye (Cat # 341831, BD Biosciences) is added to each well of the cell plate. The cell plates are further incubated for 2 hrs at room temperature in the dark. The plates are loaded on a FlexStation (Molecular Devices Corporation, Sunnyville, Calif.) to read baseline fluorescence. 10 μl of 125-250 μM PDE IV specific inhibitor Ro20-1724 is added to half of the sample wells of each clone, and 10 μl vehicle is added to the other half of the sample wells as controls. Cell plates are incubated at room temperature for 30 minutes, and loaded on a FlexStation for readings. The ratio between the wells with or without Ro20-1724 is calculated, and clones with the ratio larger than 2 are good cell line candidates for PDE IV assay.

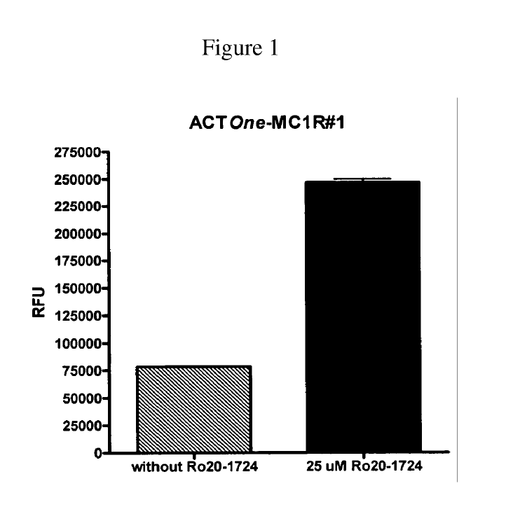

Fluorescence intensity of ACTOne-MC1R#1 (a cell line selected for PDE IV assay) in the presence and absence of Ro20-1724 using ACTOne Membrane Potential Dye is shown in FIG. 1.

### Example 3

### Identification of Agents that Modulate PDE Activity

Compounds may be screened for their ability to function as agents for the modulation of PDE activity. A cell prepared according to the present invention may be contacted with a compound and PDE activity may be assayed. As an example, stable cell lines expressing a genes encoding a CNG channel protein and a GPCR of interest can be obtained (Ausuebl et al., Current Protocols in Molecular Biology, (2001) John Wiley & Sons) and from the example above. The GPCR gene is expressed exogenously.

Before the assay, all cells are harvested when they reach 80-90% confluence or less, and they should not be overgrown. Culture medium of the transfected cells was removed and replaced with a volume of Dulbecco's Phosphate Buffers Saline without calcium and magnesium (DPBS) to adequately cover and wash the cells. The DPBS was removed and a sufficient volume of 1× trypsin-EDTA was added to just cover the cells (i.e. 1 ml for a 10 cm dish, 2 ml for a T75 flask, and 5 ml for a T150 flask). The plate was agitated to make sure the cells were equally covered with the solution. The cells were trypsinized at room temperature for about 5 min. After 5 min, the cells were examined to ensure that they were detached from the dish or flask. Gentle tapping of the dish/flask may aid in the process. Sufficient serum-containing medium were added to an appropriate volume and the medium was pipetted up and down through a serological pipette for 4 times to obtain a single cell suspension. A portion of the cells were counted with a hemocytometer. Cells are diluted in their appropriate medium at 7×10⁵ cells/ml. 100 μl/well of cell suspension is added to 96-well plates or 20 μl/well is added to 384-well plates 16-24 hours before use. For cells with HEK293 background, poly-D-lysine-coated plates are recommended. Optimal assay conditions using standard fluorescence plate readers require a confluent monolayer of cells prior to the assay. The number of cells used per well will depend upon a number of conditions including the cell line and the instrument being used to make the reading.

Cell plates are removed from the incubator after overnight incubation and examined under microscope. A confluent lawn of consistently spread cells is observed. An equal volume of 1× Membrane Potential Dye Solution (BD Biosciences, Rockville, Md.) is then added to each well (e.g. 100 μl to 100 μl culture medium/well for 96-well plates, or 20 μl to 20 μl culture medium/well for 384-well plates). Do not remove the culture supernatant prior to adding the 1× Dye Solution. The cell plates are further incubated for 2 hrs at room temperature in the dark.

Libraries of PDE inhibitors can be obtained and diluted to desired concentrations for testing. As an example, PDE inhibitors Ro20-1724 and IBMX were diluted in 1×DPBS as shown in Table 1. These concentrations are 5× the expected final testing concentrations.

TABLE 1
An example of the concentrations of testing
compounds in a compound dilution plate

| | Ro20-1724 (μM) | IBMX (μM) |
|---|---|---|
| A | 2500 | 5000 |
| B | 750 | 1500 |
| C | 250 | 500 |
| D | 75 | 150 |
| E | 25 | 50 |
| F | 7.5 | 15 |
| G | 2.5 | 5 |
| H | 0 | 0 |

Membrane potential assays are performed as described in the manual for ACTOne Membrane Potential Dye Kit (BD Biosciences, Rockville, Md.). Test compounds can be added on-line (some fluorescence plate readers also have fluid addition module, and therefore compound addition and fluorescence intensity reading can happen simultaneously. i.e. FLIPR and FlexStation) or off-line (other liquid handling equipments are used for compound addition). Test compounds in 1×DPBS was added to the cell plates at 50 μl/well for 96-well plates (250 μl total well volume after addition) or 10 μl/well for 384-well plates (50 μl total well volume after addition). For example, the final concentrations of PDE inhibitors Ro20-1724 and IBMX used are listed in Table 2.

TABLE 2
An example of the final testing concentrations
of PDE inhibitors in a cell assay plate

| | Ro20-1724 (μM) | IBMX (μM) |
|---|---|---|
| A | 500 | 1000 |
| B | 150 | 300 |
| C | 50 | 100 |
| D | 15 | 30 |
| E | 5 | 10 |
| F | 1.5 | 3 |
| G | 0.5 | 1 |
| H | 0 | 0 |

Cell plates are loaded into a FLIPR, FLEXstation, or other fluorescence microplate reader to read fluorescence intensity before and after compound addition. The settings on FLIPR are described in the manuals from the manufacturer. For FLEXstation and other fluorescence microplate readers, wavelengths close to the maxima of absorption and emission of the dye are used: for example, 530 nm excitation, 550 auto cut-off, and 565 nm emission.

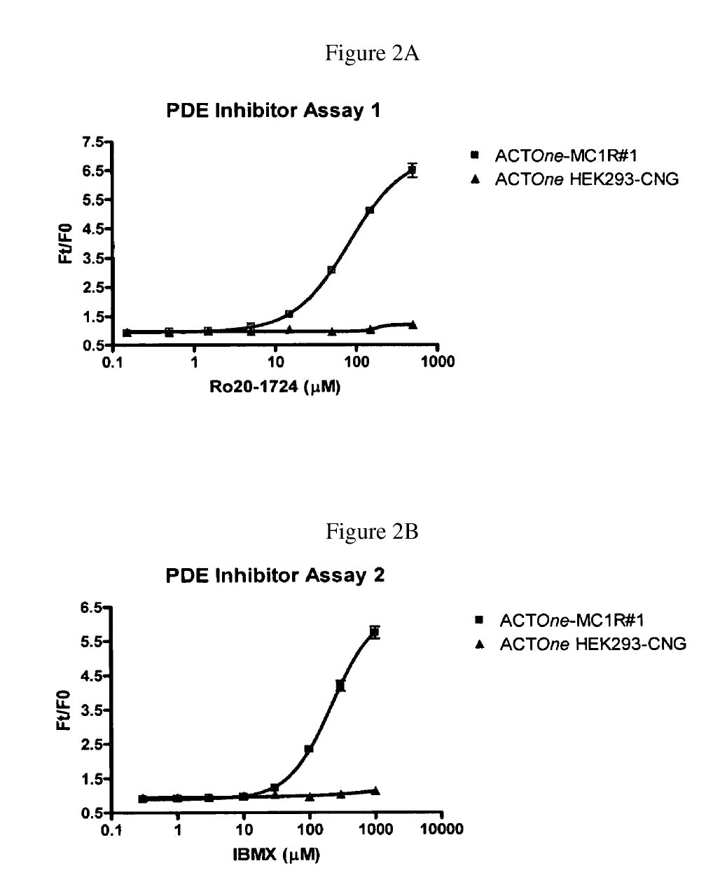

Dose response curves of PDE inhibitors Ro20-1724 and IBMX in ACTOne-MC1R#1 cell line were overlaid as shown in FIGS. 2A and 2B. ACTOne HEK293-CNG cells were used as a negative control.

In the kinetic assay, the compound is added at 20^{th} sec with 50 μl/well for 96-well plates (250 μl total well volume after addition) or 10 μl/well for 384-well plates (50 μl total well volume after addition). The kinetic curves were recorded by FlexStation at 3 sec interval. Multiple fluorescence traces were overlaid as shown in FIG. 2C.

Two more cell lines, ACTOne-IRES-A2b#2 and ACTOne-TSHR#5, were tested using the similar approach described above. The procedure for constructing these cell lines were described in Example 1.

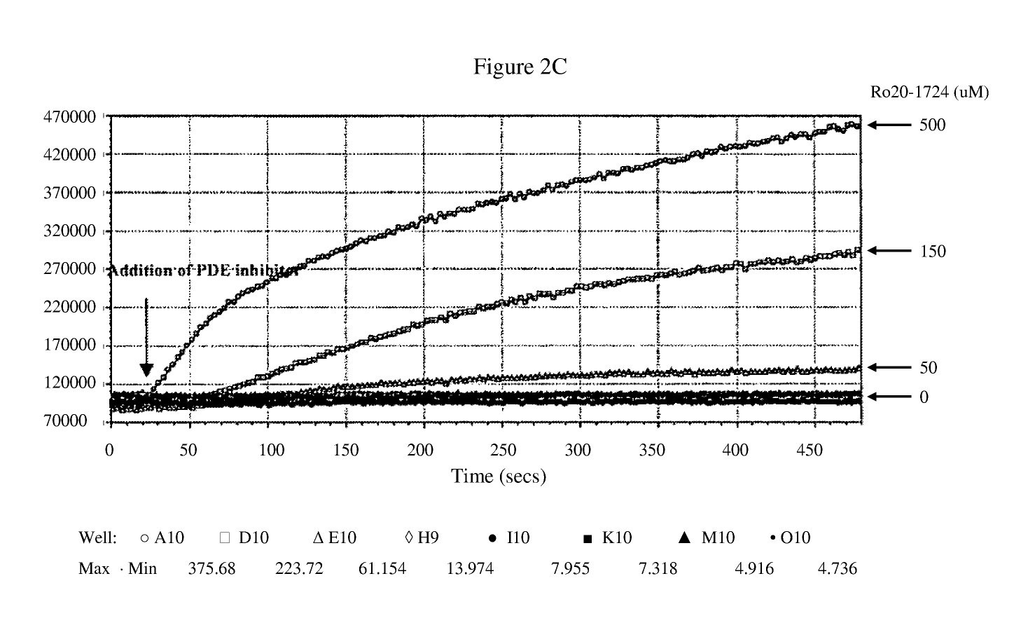

FIGS. 3A and 313 show the dose response curves of Ro20-1724 and IBMX in ACTOne-TSHR#5 cells. Fluorescence intensity counts obtained before and 30 minutes after compound addition were used for data analysis.

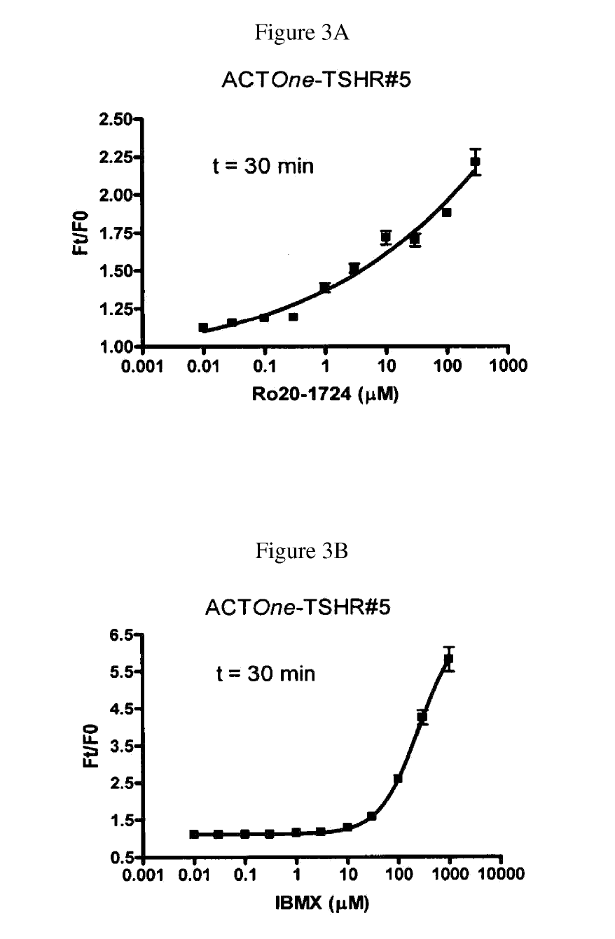

FIG. 4 shows the dose response curves of Ro20-1724 and IBMX in ACTOne-IRES-A2b#2 cell line. Fluorescence intensity counts obtained before and 30 minutes after compound addition were used for data analysis. It has been reported that IBMX also functions as an antagonist of A1b receptor in addition to a PDE inhibitor, and therefore the PDE inhibition activity of IBMX can not be detected by this cell line.

### Example 4

### Specificity of the PDE Assay

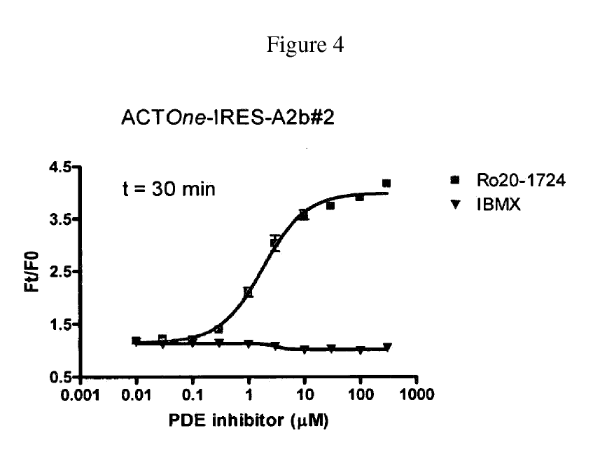

It has been reported that PDE4 is a dominant isozyme in HEK293 cells. In order to analyze the specificity of the PDE assay, different PDE inhibitors (Ro20-1724, Rolipram, Etazolate (PDE IV inhibitors); 8-methoxymethyl-3-isobuty-1-m (PDE I inhibitor); Zaprinast (cGMP specific PDE inhibitor); Quazinon (PDE III inhibitor); IBMX (pan PDE inhibitor)) were purchased from Sigma. They were diluted at different concentrations in 1×DPBS and the assays were performed the same as above using the ACTOne-IRES-A2b#2 cell line. Briefly, the cells were plated on 384-well PDL coated plate. The density of the cells was 14,000 cells per well. The cells were allowed to attach and grow overnight. On the 2^{nd} day, the cells were loaded with 1× ACTOne Membrane Potential dye and incubated at room temperature for 2 hours in the dark. Different concentrations of PDE inhibitors were prepared in 1×DPBS. Two fluorescence intensity readings were obtained before and 30 minutes after compound addition. FIG. 5 shows dose response curves of different PDE inhibitors on the ACTOne-IRES-A2b#2 cell line. PDE IV inhibitors were detected as expected while other inhibitors gave negative signals, indicating high specificity of this assay.

### Example 5

### Endpoint and Kinetic Assays with Calcium-Sensitive Dye

The PDE assay can also be carried out on the cell lines described above using calcium sensitive dyes.

The cells were harvested and plated on poly-D lysine coated plates. The density of the cells was 14,000 cells per well for 384 well plates and 70,000 cells per well for 96 well plates. The cells were allowed to attach and grow overnight. On the second day, the cells were loaded with BD™ PBX calcium assay kit (BD Biosciences, Rockville, Md.) and incubated at 37° C. for 1 hour. Afterward, the cells were left at room temperature for 30 minutes. During incubation, test compounds were prepared by dissolving in 1×HBSS containing 15 mM CaCl₂.

Dye loaded cell plates are then loaded into a FLIPR, FLEXstation, or other fluorescence microplate reader and assayed per fluorescence microplate reader instructions.

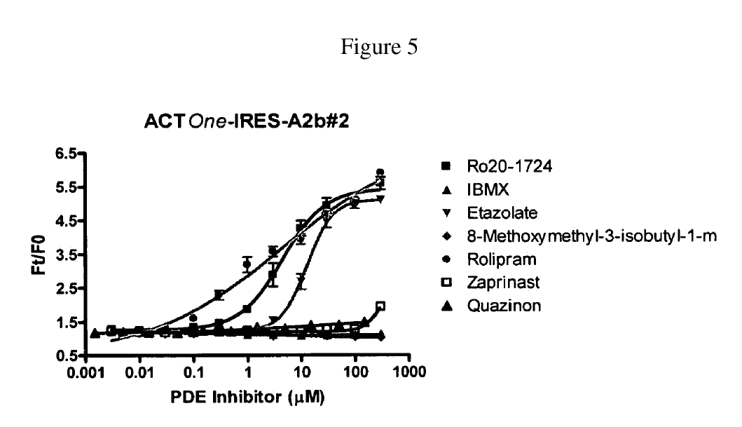

Dose response curves of Ro-20-1724 and IBMX (FIGS. 6A and 6B) and multiple fluorescence traces (FIG. 6C) in response to the various doses of compounds in ACTOne-MC1R#1 were overlaid as shown in FIG. 6.

### Example 6

### Protocol to Establish PDE2A Stable Cells (Method 1)

PDE2A cells may be established using the following method.

The day before transfection, plate 5 million 293FT cells on a 10 cm dish with 10 ml of DMEM, 10% FBS. The following day, remove the culture medium from 293FT cells and replace it with 5 ml of DMEM, 10% FBS. Dilute 3 μg of pLenti6-PDE2A and 9 μg optimized packaging mix (Invitrogen) in 1.5 ml of Opti-MEM (Invitrogen). Dilute 36 μl of Lipofectamine 2000 (Invitrogen) in 1.5 ml of Opti-MEM and incubate for 5 min. at room temperature. Combine the solutions from step 1 (DNA) and step 2 (Lipofectamine) and incubate at room temperature for 20 min. to allow DNA-Lipofectamine complexes to form. Add DNA-Lipofectamine 2000 complexes directly to 293FT cells and mix gently by rocking back and forth. Incubate cells overnight in a 37° C.-CO₂ incubator.

On day 2, change media to 6 ml of fresh DMEM with 10% FBS, and incubate in a 37° C.-CO₂ incubator for another 48 hours.

Split ACTOne-TSHR#112 cell line at 2×10⁵ cells per well in a 6-well plate with 2 mls DMEM, 10% FBS.

On day 4, harvest viral particle by transfer medium from Day 2 (293FT posttransfection) to a 15 ml conical tube, and centrifuge at 3000 rpm for 15 min. After centrifugation, filter the viral supernatant through a sterile 0.45 μm low protein binding filter (Millipore). Take portion of the viral supernatant, prepare 10-fold serial dilution in DMEM with 10% FBS, ranging from 10⁻² to 10⁻⁴. Aliquot the rest of viral supernatant (not diluted) and store in −80° C. for future use.

Remove the medium on ACTOne-TSHR#112 cells, add 2 ml diluted viral supernatant. Add Polybrene to each well to a final concentration of 6 μg/ml. Swirl the plate gently and incubate at 37° C. overnight.

On day 5, replace the medium on ACTOne-TSHR#112 cells with fresh DMEM with 10% FBS and 1× Non-essential amino acids (Invitrogen). Incubate in a 37° C.-CO₂ incubator for 24 hours.

On day 6, transfer the infected cells to 10 cm dishes with proper dilutions. Select the cells with 10 ml of DMEM-10% FBS supplemented with 250 μg/ml G418, 1 μg/ml Puromycin and 6 μg/ml blasticidin. In about 2-3 weeks, colonies will be observed. Pick up single colonies and transfer to 24 well-plate containing 1 ml of DMEM-10% FBS supplemented with 250 μg/ml G418, 1 μg/ml Puromycin and 5 μg/ml blasticidin.

Assay Protocol to Measure PDE2A Inhibitor with BD Membrane Potential Dye Kit

The cells are plated for assays as follows.

Remove the culture medium and replace it with a volume of Dulbecco's Phosphate Buffers Saline without calcium and magnesium (DPBS) to adequately cover and wash the cells. Remove DPBS. Add a sufficient volume of 1× trypsin-EDTA to just cover the cells (i.e. 1 ml for a 10 cm dish, 2 ml for a T75 flask, and 5 ml for a T150 flask). Rock the plate to make sure the cells are equally covered with the solution. Trypsinize the cells at room temperature for ˜5 min. After 5 min, check the cells to ensure that they are coming off the dish/flask. Gentle tapping of the dish/flask may aid in the process. Add enough growth medium to give a volume of ˜10 ml and pipette the medium up and down through a 10 ml serological pipette for ˜4 times to obtain a single cell suspension. Count a portion of the cells with a hemocytometer. All cells need to be harvested when they reach 80-90% confluence or less at all times.

For cells with HEK293 background, poly-D-lysine-coated plates are recommended. Optimal assay conditions require a confluent monolayer of cells prior to the assay. It is recommended to plate out cells at 70,000 cells/well for 96-well plates and 14,000 cells/well for 384-well plates. The cells are typically diluted in their appropriate medium at 7×10⁵/ml. Add 100 μl/well of cell suspension to 96-well plates or 20 μl/well to 384-well plates 16-24 hours before use. The number of cells used per well will depend upon a number of conditions including the cell line and the instrument being used to make the reading.

Allow cells to attach and grow overnight. Observe cells microscopically the following day. A confluent lawn of consistently spread cells should be observed. If cells are obviously unhealthy or over-confluent, do not use. Gaps between cells may result in higher well-to-well variability.

The following procedure may be used for loading dye with 96-well or 384-well plates. Thaw 1× potentiometric dye solution (ActoOne™ Membrane Potential Dye Kit, BD Biosciences, BioImaging Systems, Rockville, Md.). Remove cell plates from incubator and add an equal volume of 1× Dye Solution to each well (e.g. 100 μl to 100 μl culture medium/well for 96-well plates, or 20 μl to 20 μl culture medium/well for 384-well plates), without removing the culture supernatant. Incubate cell plates with the dye for 2 hrs at room temperature in the dark.

To prepare compound plates, dilute 30 mM EHNA and 180 mM Ro 20-1724 with 1×PBS at the concentrations shown in Table 3. EHNA inhibits PDE2 and Ro 20-1724 inhibits PDE4.

TABLE 3
An example of 5x concentrations of Ro 20-
1724 and EHNA in a 96-well compound plate

| Sample # | 1 | 2 | 3 | 4 | 5 | 6 | 7 | 8 | 9 | 10 | 11 | 12 |
|---|---|---|---|---|---|---|---|---|---|---|---|---|
| Ro 20-1724 (μM) | 150 | | | | | | | | | | | |
| EHNA (μM) | 500 | 150 | 50 | 15 | 5 | 1.5 | 0.5 | 0.15 | 0.05 | 0.015 | 0.005 | 0 |

Dilute 10 mM Bay 60-7550 and 180 mM Ro 20-1724 with DMSO at the concentrations shown in Table 4. Bay 60-7550 inhibits PDE2 and Ro 20-1724 inhibits PDE4.

TABLE 4
An example of 100x concentrations of Ro 20-1724
and Bay 60-7550 in a 96-well compound plate

| Sample # | 1 | 2 | 3 | 4 | 5 | 6 | 7 | 8 | 9 | 10 | 11 | 12 |
|---|---|---|---|---|---|---|---|---|---|---|---|---|
| Ro 20-1724 (mM) | 1 | | | | | | | | | | | |
| Bay 60-7550 (μM) | 1,000 | 300 | 100 | 30 | 10 | 3 | 1 | 0.3 | 0.1 | 0.03 | 0.01 | 0 |

Further dilute the compounds 1:20 with 1×DPBS in compound plates. At this step, the compound concentrations are 5× testing concentrations. DMSO final concentration in assay wells should not exceed 1.5%.

The assays are performed as follows.

Add the test compounds in 1×DPBS to the cell plates at 50 μl/well for 96-well plates (250 μl total well volume after addition) or 10 μl/well for 384-well plates (50 μl total well volume after addition). The final concentrations of the compounds used are listed in Table 5 (EHNA) and 6 (Bay 60-7550).

TABLE 5
An example of the final testing concentrations of
Ro 20-1724 and EHNA in a 96-well cell assay plate

| Sample # | 1 | 2 | 3 | 4 | 5 | 6 | 7 | 8 | 9 | 10 | 11 | 12 |
|---|---|---|---|---|---|---|---|---|---|---|---|---|
| Ro 20-1724 (μM) | 30 | | | | | | | | | | | |
| EHNA (μM) | 100 | 30 | 10 | 3 | 1 | 0.3 | 0.1 | 0.03 | 0.01 | 0.003 | 0.001 | 0 |

TABLE 6
An example of the final testing concentrations of Ro
20-1724 and Bay 60-7550 in a 96-well cell assay plate

| Sample # | 1 | 2 | 3 | 4 | 5 | 6 | 7 | 8 | 9 | 10 | 11 | 12 |
|---|---|---|---|---|---|---|---|---|---|---|---|---|
| Ro 20-1724 (μM) | 10 | | | | | | | | | | | |
| Bay 60-7550 (nM) | 10,000 | 3,000 | 1,000 | 300 | 100 | 30 | 10 | 3 | 1 | 0.3 | 0.1 | 0 |

The assays are performed on a FlexStation, using the following wavelength parameters: Excitation: 530 nm; AutoCutoff: on (550 nm); Emission: 565 nm. The fluorescence signal from the assay is sufficiently stable to allow endpoint assays. When performing an endpoint assay, two readings are obtained, one prior to the addition of a test compounds (F₀), and the other, 15 min after the addition of the compounds (Ft). Calculate F/F₀ for data analysis.

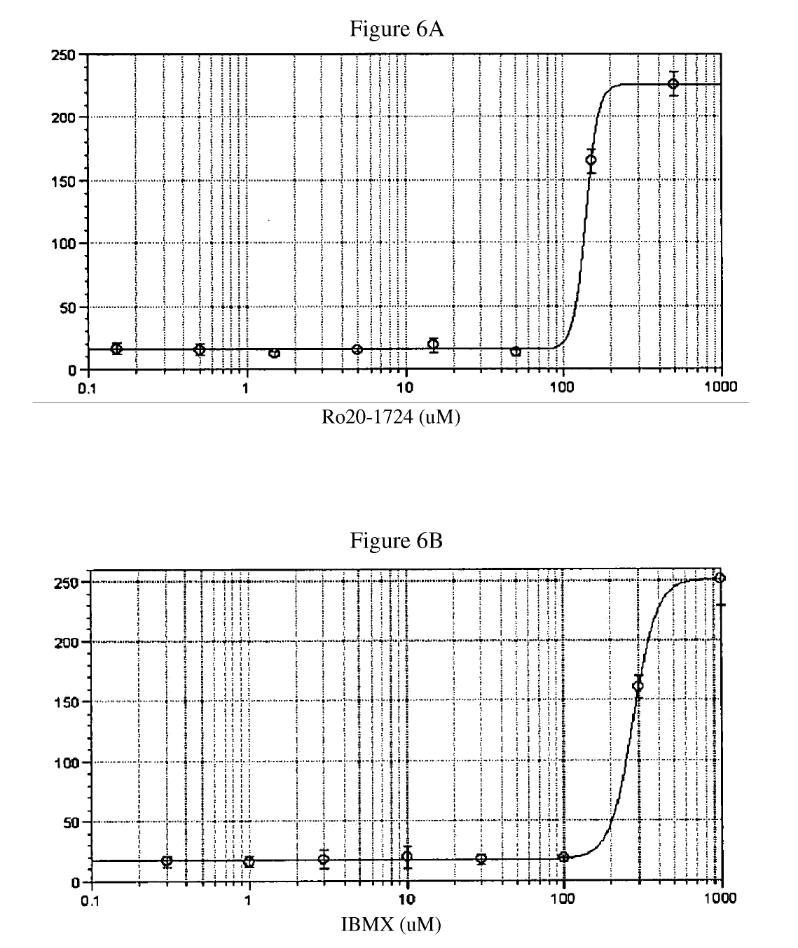

Exemplary results are shown in FIG. 7. FIG. 7 is a graph showing the dose response curves of EHNA (A) and Bay 60-7550 (B) in PDE2A expressing ActOne-TSHR#112 cells, using a conventional potentiometric dye.

### Example 7

### Protocol to Establish PDE2A Stable Cells (Method 2)

PDE2A cells may also be established using the following method.

Split ASC0200 cells into 6 well-plate with 70-80% confluence (or follow Invitrogen protocol). For each well, the cells are transfected with A) 0.6 μg pEAK10-TSHR plus 1.8 μg pEAK10-PDE2A (1:3); B) 1.2 μg pEAK10-TSHR plus 1.2 μg pEAK 10-PDE2A (1:1); C) 1.8 μg pEAK10-TSHR plus 0.6 μg pEAK10-PDE2A (3:1). About 18-22 hrs later, split the transfected cells into 100 mm dishes. Each well goes to 2 dishes. Dilute the cells properly. At the same time, select the cells with DMEM—10% FBS supplemented with 250 μg/ml G418 and 2 μg/ml Puromycin. Change medium if there are too many dead cells. In about 2-3 weeks, the colonies will be observed. Pick up the single colonies and transferred into 24 well-plate containing 1 ml of DMEM-10% FBS supplemented with 250 μg/ml G418 and 1 μg/ml Puromycin.

Assay Protocol to Measure PDE2A Inhibitor with BD Membrane Potential Dye Kit

The cells are plated for assays as follows.

Remove the culture medium and replace it with a volume of Dulbecco's Phosphate Buffers Saline without calcium and magnesium (DPBS) to adequately cover and wash the cells. Remove DPBS. Add a sufficient volume of 1× trypsin-EDTA to just cover the cells (i.e. 1 ml for a 10 cm dish, 2 ml for a T75 flask, and 5 ml for a T150 flask) Rock the plate to make sure the cells are equally covered with the solution. Trypsinize the cells at room temperature for ˜5 min. After 5 min, check the cells to ensure that they are coming off the dish/flask. Gentle tapping of the dish/flask may aid in the process. Add enough growth medium to give a volume of ˜10 ml and pipette the medium up and down through a 10 ml serological pipette for ˜4 times to obtain a single cell suspension. Count a portion of the cells with a hemocytometer. All cells need to be harvested when they reach 80-90% confluence or less at all times. Do not grow the cells.

For cells with HEK293 background, poly-D-lysine-coated plates are recommended. Optimal assay conditions require a confluent monolayer of cells prior to the assay. It is recommended to plate out cells at 70,000 cells/well for 96-well plates and 14,000 cells/well for 384-well plates. The cells are typically diluted in their appropriate medium at 7×10⁵/ml. Add 100 μl/well of cell suspension to 96-well plates or 20 μl/well to 384-well plates 16-24 hours before use. The number of cells used per well will depend upon a number of conditions including the cell line and the instrument being used to make the reading. Allow cells to attach and grow overnight. Observe cells microscopically the following day. A confluent lawn of consistently spread cells should be observed. If cells are obviously unhealthy or over-confluent, do not use. Gaps between cells may result in higher well-to-well variability.

The dye is then loaded as follows using 96-well or 384-well plates. Thaw 1× potentiometric dye solution as in Example 6. Remove cell plates from incubator and add an equal volume of 1× Dye Solution to each well (e.g. 100 μl to 100 μl culture medium/well for 96-well plates, or 20 μl to 20 μl culture medium/well for 384-well plates), without removing the culture supernatant. Incubate cell plates with the dye for 2 hrs at room temperature in the dark.

To prepare compound plates, dilute 10 mM Bay 60-7550 and 180 mM Ro 20-1724 with DMSO at the concentrations shown in Table 7. Bay 60-7550 inhibits PDE2 and Ro 20-1724 inhibits PDE4.

TABLE 7
An example of 100x concentrations of Ro 20-1724
and Bay 60-7550 in a 96-well compound plate

| Sample # | 1 | 2 | 3 | 4 | 5 | 6 | 7 | 8 | 9 | 10 | 11 | 12 |
|---|---|---|---|---|---|---|---|---|---|---|---|---|
| Ro 20-1724 (mM) | 3 | | | | | | | | | | | |
| Bay 60-7550 (μM) | 1,000 | 300 | 100 | 30 | 10 | 3 | 1 | 0.3 | 0.1 | 0.03 | 0.01 | 0 |

Further dilute the compounds 1:20 with 1×DPBS in compound plates. At this step, the compounds concentrations are 5× testing concentrations. The DMSO final concentration in assay wells should not exceed 1.5%.

The assays may be performed as follows.

Add the test compounds in 1×DPBS to the cell plates at 50 μl/well for 96-well plates (250 μl total well volume after addition) or 10 μl/well for 384-well plates (50 μl total well volume after addition). The final concentrations of the compounds used are listed in Table 8 (Bay 60-7550).

TABLE 8
An example of the final testing concentrations of Ro
20-1724 and Bay 60-7550 in a 96-well cell assay plate

| Sample # | 1 | 2 | 3 | 4 | 5 | 6 | 7 | 8 | 9 | 10 | 11 | 12 |
|---|---|---|---|---|---|---|---|---|---|---|---|---|
| Ro 20-1724 (μM) | 30 | | | | | | | | | | | |
| Bay 60-7550 (nM) | 10,000 | 3,000 | 1,000 | 300 | 100 | 30 | 10 | 3 | 1 | 0.3 | 0.1 | 0 |

The assays are performed on a FlexStation, using the following wavelength parameters: Excitation: 530 nm; AutoCutoff: on (550 nm); Emission: 565 nm.

The fluorescence signal from the assay is sufficiently stable to allow endpoint assays. When performing an endpoint assay, two readings are obtained, one prior to the addition of a test compounds (F₀), and the other, 30 min after the addition of the compounds (Ft). Calculate F/F₀ for data analysis.

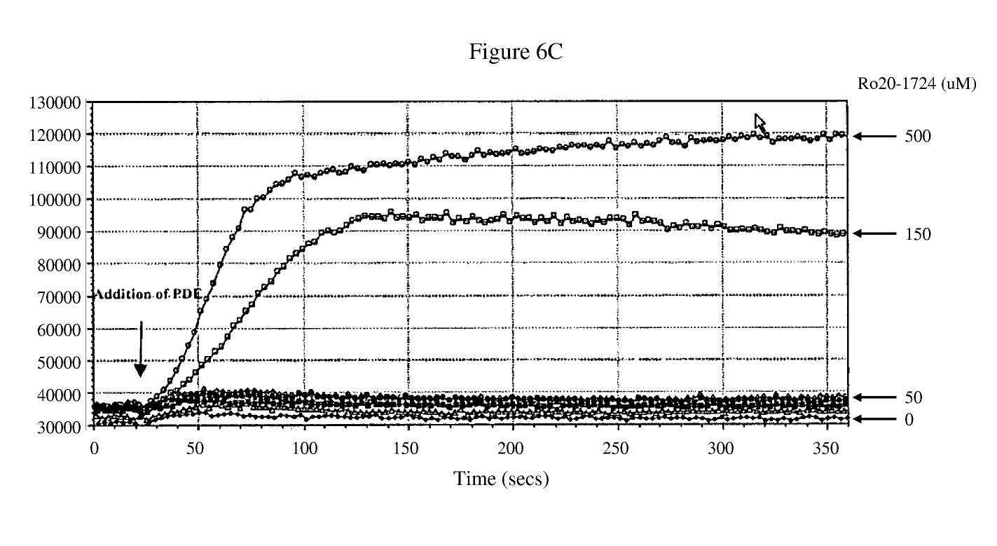

Exemplary results are shown in FIG. 8. FIG. 8 is a graph showing the dose response curve of Bay 60-7550 in PDE2A and TSHR expressing ASC0200 cells, using a conventional potentiometric dye.

Although the present invention has been described in detail with reference to examples above, it is understood that various modifications can be made without departing from the spirit of the invention. Accordingly, the invention is limited only by the following claims. All cited patents, patent applications and publications referred to in this application are herein incorporated by reference in their entirety.

1-80. (canceled)

81. A method for identifying a compound that modulates phosphodiesterase (PDE) activity, comprising:
(a) providing a cell that expresses:
a phosphodiesterase (PDE) protein;
a cyclic nucleotide gated (CNG) channel protein; and
an exogenous G protein coupled receptor (GPCR) protein that increases the level of cyclic nucleotide production in the absence of external stimulation of intracellular cyclic nucleotide production;
wherein the increased level of cyclic nucleotide produced by the said exogenous GPCR protein does not activate said CNG channel in the absence of either external stimulation of intracellular cyclic nucleotide production, an inhibitor of said PDE, or both;
(b) contacting said cell, in the absence of external stimulation of intracellular cyclic nucleotide production, with a compound that putatively modulates the activity of said PDE; and
(c) measuring activity of said CNG channel, wherein the activity of said CNG channel is indicative of changes in cellular concentration of a cyclic nucleotide; thereby identifying whether said compound modulates the activity of said PDE.

82. The method of claim 81, wherein said PDE is exogenous.

83. The method of claim 82, wherein an endogenous phosphodiesterase (PDE) of said cell is suppressed by an inhibitor specific to said endogenous PDE.

84. The method of claim 81, wherein said cell is selected from the group consisting of insect cells, amphibian cells, yeast cells, and mammalian cells.

85. The method of claim 81, wherein said cell is selected from the group consisting of HEK-293, CHO, Hela and BHK.

86. The method of claim 85, wherein said cell is HEK-293.

87. The method of claim 81, wherein said activity is measured using an intracellular cyclic nucleotide indicator selected from the group consisting of a membrane potential indicator, a cation-sensitive indicator, a FRET-based indicator, and a cAMP-responsive element (CRE).

88. The method of claim 87, wherein said indicator is selected from the group consisting of a fluorescent indicator and a luminescent indicator.

89. The method of claim 87, wherein said indicator is a cation-sensitive indicator selected from the group consisting of a calcium-sensitive indicator, a sodium-sensitive indicator and a potassium-sensitive indicator.

90. A method of claim 81, further comprising: (d) comparing activation of said CNG channel to activation of said CNG channel in the absence of said compound, wherein a difference in activation of said CNG channel indicates the compound inhibits the activity of said PDE.

91. A method of claim 81, further comprising: (d) comparing activation of said CNG channel to activation of said CNG channel by a known inhibitor of said PDE, wherein a similar pattern of activation of said CNG channel indicates said compound inhibits the activity of said PDE.

92. A cell comprising:
a phosphodiesterase (PDE) protein;
a cyclic nucleotide gated (CNG) channel; and
an exogenous G protein coupled receptor (GPCR) protein that increases the level of intracellular cyclic nucleotide production in the absence of external stimulation of intracellular cyclic nucleotide production,
wherein activation of said CNG channel is not detected in the absence of an inhibitor of said PDE and wherein activation of said CNG channel is detected in the presence of an inhibitor of said PDE.

93. The cell of claim 92, wherein said PDE is exogenous.

94. The cell of claim 92, wherein an endogenous phosphodiesterase (PDE) of said cell is suppressed by an inhibitor specific to said endogenous PDE.

95. The cell of claim 92, wherein said cell is selected from the group consisting of HEK-293, CHO, Hela and BHK.
